(19) United States
(12) Patent Application Publication
(10) Pub. No.: US 2023/0081382 A1
(43) Pub. Date: Mar. 16, 2023

(54) Ex Vivo Method for Removal of RCDP

(71) Applicant: NHS Blood & Transplant, Bristol (GB)

(72) Inventors: David Anstee, Bristol (GB); Tosti Mankelow, Bristol (GB); Sara Trompeter, Bristol (GB)

(21) Appl. No.: 17/798,356

(22) PCT Filed: Feb. 10, 2021
(86) PCT No.: PCT/GB2021/050298

§ 371 Date: Aug. 9, 2022

(30) Foreign Application Priority Data
Feb. 10, 2020 (GB) 2001795.0

(51) Int. Cl. A61K 35/16; A61M 1/34; A61M 1/36; A61M 1/38; A61P 7/04; B01D 15/38

(52) U.S. Cl. CPC A61K 35/16; A61M 1/3496; A61M 1/3687; A61M 1/38; A61P 7/04; B01D 15/3809; A61M 2202/0429; A61M 2202/0435; A61M 2202/0415

(57) ABSTRACT

A method of decreasing hypercoagulability and/or increasing plasma clotting time comprising removing red cell-derived particles (RCDP) from plasma.

### FIELD OF THE INVENTION

The present invention relates to methods of decreasing hypercoagulability, methods of increasing plasma clotting time, and methods of treating a haemoglobinopathy, haemolytic anaemia or an erythrocyte membrane disorder. The present invention also relates to plasma obtained or obtainable by said methods. The present invention also relates to affinity columns and apheresis devices and their use in said methods.

### BACKGROUND TO THE INVENTION

Haemoglobinopathies are hereditary conditions involving an abnormality in the structure of haemoglobin. More than 1,000 mutations have been identified that result in either haemoglobin variants or thalassemias. Common haemoglobinopathies include sickle-cell disease.

Sickle Cell Disease (SCD) is a group of inherited blood disorders, including sickle cell anaemia (SCA), HbSC and HbSβ-thalassaemia, which results in an abnormality in the haemoglobin found in red blood cells. The incidence is estimated to be between 300,000 and 400,000 neonates globally each year, the majority in sub-Saharan Africa. Common acute complications are acute pain events, acute chest syndrome and stroke. Chronic complications (including chronic kidney disease) can damage all organs (Kato, G. J., et al., 2018. Nature Reviews Disease Primers, 4, p.18010).

SCD is characterised by haemolytic anaemia, hypercoagulability and inflammation. This complex pathology results from the inheritance of homozygosity for a single base change (Glu to Val at codon 6) in the gene encoding the haemoglobin subunit β (HBB) which creates a haemoglobin molecule capable of polymerisation at low oxygen concentrations. Erythrocytes that contain mostly haemoglobin polymers assume a sickled form and are prone to haemolysis.

Advances in general medical care, early diagnosis and comprehensive treatment have led to substantial improvements in the life expectancy of individuals with SCD in high-income countries as almost all patients survive beyond 18 years of age. However, even with the best of care, life expectancy is still reduced by ˜30 years, routine and emergency care for individuals with SCD have great financial costs, the quality of life often deteriorates during adulthood, and the social and psychological effects of SCD on affected individuals and their families remain underappreciated. Furthermore, most of these advances have not reached low-income countries (Kato, G. J., et al., 2018. Nature Reviews Disease Primers, 4, p.18010).

Thus, there is a need for new methods of treating SCD and/or alleviating one or more symptoms of SCD, such as hypercoagulability.

### SUMMARY OF THE INVENTION

The inventors have surprisingly found that red cell-derived particles (RCDP) are a major contributor to hypercoagulability in SCD and that removal of RCDP from the circulation of SCD patients can increase the plasma clotting time and decrease hypercoagulability.

The inventors have surprisingly found a heterogeneous population of RCDP in SCD patients. The inventors have surprisingly found that right-side out erythrocyte-derived macrovesicles with exposed phosphatidylserine may significantly contribute to the hypercoagulative state in SCD patients.

Thus, removal of RCDP from the circulation of SCD patients offers a novel therapeutic treatment capable of improving the quality of life of patients with SCD.

In one aspect, the present invention provides a method of decreasing hypercoagulability and/or increasing plasma clotting time comprising removing red cell-derived particles (RCDP) from plasma isolated from a subject to provide modified plasma.

In another aspect, the present invention provides a method of decreasing hypercoagulability, increasing plasma clotting time, and/or treating a haemoglobinopathy, haemolytic anaemia, or an erythrocyte membrane disorder in a subject, wherein the method comprises removing RCDP from plasma isolated from the subject to provide modified plasma. The modified plasma may be administered to the subject.

In another aspect, the present invention provides a method of decreasing hypercoagulability, increasing plasma clotting time, and/or treating a haemoglobinopathy, haemolytic anaemia, or an erythrocyte membrane disorder in a subject, wherein the method comprises:
(i) isolating plasma from the subject;
(ii) removing RCDP from the plasma to provide modified plasma; and
(iii) administering the modified plasma to the subject.

In another aspect, the present invention provides modified plasma obtained or obtainable by a method of the present invention.

In another aspect, the present invention provides plasma which is substantially free of RDCP or substantially free of one or more RCDP subpopulation.

In another aspect, the present invention provides a molecule which specifically binds one or more markers on the surface of RCDP, wherein the molecule is immobilised.

In another aspect, the present invention provides a bead which specifically binds one or more markers on the surface of RCDP.

In another aspect, the present invention provides an affinity column which specifically binds one or more markers on the surface of RCDP.

In another aspect, the present invention provides an apheresis device or an apheresis kit comprising a molecule, a bead, and/or an affinity column which specifically binds one or more markers on the surface of RCDP.

In another aspect, the present invention provides for use of a molecule, a bead, an affinity column, or an apheresis device or kit of the present invention, for removing RCDP, for increasing plasma clotting time, and/or for decreasing hypercoagulability.

In another aspect, the present invention provides for use of a molecule, an affinity column, a bead, or an apheresis device or kit of the present invention, in a method of the present invention.

### DESCRIPTION OF DRAWINGS

FIG. 1—ImageStream analysis of BRIC256-Alexa Fluor 647 and BRIC163- Alexa Fluor 488 stained SCD platelet free plasma (PFP)

To remove platelets SCD plasma was centrifuged 2000 g for 15 minutes at room temperature with no brake followed by two further centrifugations (2500 g for 20 minutes at room temperature), with the pellet discarded after each step. SCD PFP was processed and stained with BRIC256-Alexa Fluor 647 and BRIC163-Alexa Fluor 488. (A) Analysis of all RCDP detected (i) revealed the presence of BRIC256 single stained particles (right-side out), BRIC163 stained particles (inside-out) and dual stained particles (membrane fragments). Further analysis split the RCDP into (ii) low scatter (small size) and (iii) high scatter (large size). Images of (B) low scatter and (C) high scatter of (i) BRIC256^{+ve}, (ii) dual^{+ve} and (iii) BRIC163^{+ve} RCDPs. (D) Numbers of (i) BRIC256^{+ve}, (ii) BRIC163^{+ve} and (iii) dual^{+ve} events found in SCD plasma from patients in Crisis (circle, n=10), Steady State (triangle, n=11) and from healthy individuals (square, n=5).

Each data point represents one sample with the black bars showing the mean number of events in each cohort along with standard deviation. Points with the same shape and shade are identical samples. Statistically significant differences are shown.

FIG. 2—Size determination of RCDP present in SCD plasma

SCD plasma was stained with BRIC256-Alexa Fluor 647 and BRIC163-Alexa Fluor 488. High scatter events (encompassing all the MaV population) can be removed by filtration through a 1.2 μm filter. SCD plasma was analysed on an ImageStream along with size beads. (A) Plot showing the scatter intensity of 0.2 μm beads, 0.5 μm beads, 1 μm beads, Erythrocyte storage induced microvesicle, SCD plasma RCDP (low scatter), 1.2 μm filtered SCD plasma RCDP (low scatter) and SCD plasma macrovesicles (MaV) (high scatter) on an ImageStream flow cytometer. (B) Samples were run on a standard flow cytometry (Navios, Beckmann Coulter) and a plot of side scatter versus forward scatter produced for (i) size beads and (ii) and (iii) BRIC256^{+ve} particles in the plasma of two SCD patients. The position of the size beads is shown on the scatter plots to illustrate the relationship between microparticles detected by the flow cytometer and size. The areas corresponding to size beads are drawn on the RCDP plot (ii) and (iii). (C) Spinning-disk confocal analysis of BRIC256+ve sorted steady state SCD samples. (i) Spinning-disk confocal image. Scale bar 5 μm. (ii) Scatter plot of size of each individual round object detected in patient samples. Line represents median values.

FIG. 3—Annexin V staining of BRIC256 positive RCDP in SCD plasma

SCD plasma stained with BRIC256-Alexa Fluor 488 and Annexin V-Alexa Fluor 647. Shown is the analysed data for (A) high scatter events and (B) low scatter events. Numbers of BRIC256^{+ve} only and BRIC256-Annexin V dual^{+ve} events found in SCD plasma from patients in crisis (circles, n=5), steady state (triangles, n=10) and from healthy individuals (squares, n=4). Each symbol represents an individual patient sample. Bars represent mean number of events in each cohort ± standard deviation. Statistically significant differences are shown. (A) There is a significant difference between the number of dual BRIC256-Annexin V^{+ve} RCDP in SCD patients plasma in crisis and healthy controls. (B) There are significant differences between BRIC256^{+ve} and BRIC256-Annexin V^{+ve} RCDP in plasma from each individual sample, regardless of source (Crisis P=0.01, Steady State P<0.0001 and Healthy P=0.4). There were significantly more BRIC256^{+ve} events in patients in crisis compared to those in steady state and healthy controls (P<0.0001). (C) Images of high scatter events showing (i) the purely dual^{+ve} MaV subpopulation and the mixed BRIC256 single and BRIC256-Annexin V dual^{+ve} smaller RCDP. Scale bars 7 μm.

FIG. 4—Transmission electron microscopy of enriched RCDP

Isolated right side RCDP (A) were stained using (i) immunogold labelled extracellular GPA or (ii) and (iii) extracellular AE-1. Scale bars indicate 50 nm (i) or 100 nm (ii and iii). (B) Inside out RCDP either (i) intracellular AE-1 (BRIC132) positive were stained with gold intracellular AE-1 (BRAC66) or (ii) intracellular GPA (BRIC163) positive were stained using gold intracellular AE-1 (BRAC66). Scale bars indicate 100 nm (i) or 50 nm (ii). (C) MaV stained using (i) immunogold labelled extracellular GPA. Boxed area corresponds to area shown in (ii). Scale bars indicate 2 μm (i) or 200 nm. Light arrows indicate immunogold particles and dark arrows indicate magnetic beads.

FIG. 5—Clotting times of PFP is affected by the presence of RCDP

RCDP were selectively removed from SCD PFP using an anti-glycophorin A antibody attached to magnetic microbeads. PFP samples were assayed before and after RCDP removal on the ImageStream using BRIC256 Alexa fluor-647 to determine removal rates. After RCDP removal the clotting times were assayed (A) and found to increase (left hand side—steady state SCD, right hand side—healthy individual). Average steady state SCD clotting times was 128.9 seconds and average healthy clotting times was 143.3 seconds. The amount of RCDP removal was: steady state—20610 events per μl or 94.1% of all RCDP present at start, healthy—5388 events per μl or 88.8% of all RCDP present at start. (B) When SCD RCDP are added to healthy PFP the clotting times are reduced (mean SCD RCDP addition to healthy PFP 3788 events per μl) (left hand side—healthy PFP with just microbeads and right hand side—healthy PFP with SCD RCDP attached to microbeads). Average clotting times for healthy PFP with beads was 145.2 seconds and 59.8 seconds for beads and SCD RCDP. (A) and (B) n=6 SCD samples and n=3 healthy samples. (C) Healthy and SCD PFP was passed through a 1.2 μm filter and samples analysed on the ImageStream to determine changes in RCDP (mean removal rates; steady state sickle—low scatter, 140 events per μl or 1.9% of all low scatter RCDP present at start, high scatter (not including MaV) 848 events per μl or 3.4% of all high scatter RCDP present at start and MaV 4835 events per μl or 95.8% of all MaV present at start and healthy low scatter 1994 events per μl or 17.5% of all low scatter RCDP present at start, high scatter (not including MaV) 209 events per μl or 0.83% of all high scatter RCDP present at start and MaV 102 events per μl or 68% of all MaV present at start) and clotting times found to increase after filtration (left hand side—steady state SCD, right hand side—Healthy). Average steady state SCD clotting times was 92.9 seconds and average healthy clotting times was 156.8 seconds. n=4 SCD samples and n=2 healthy samples.

FIG. 6—Comparison of erythrocytes and MaV found in SCD PFP when viewed by Imaging flow cytometry.

When imaged with an ImageStream, erythrocytes are clearly different from the MaV observed in plasma from sickle cell patients. (A) erythrocytes from a healthy individual look brighter and bolder compared with (B) MaV from SCD plasma. (C) side views of MaV. Scale bars (7 μm) are shown. Erythrocytes and MaV particles are of an equivalent size.

FIG. 7—Imaging flow cytometry analysis of SCD PFP before and after 1.2 μm filtration.

SCD PFP was filtered through a 1.2 μm syringe filter and stained with BRIC256-Alexa Fluor 647. (A) Analysis of low scatter particles detected (i and iii) and high scatter particles (ii and iv) pre filtration (i and ii) and post filtration (iii and iv). MaV particles are included in a separate gate in the high scatter population (Ghost). (B) Brightfield and fluorescence images of the MaV population pre filtration. (C) Table of number of events (per 105 GPA⁺ events) pre and post filtration, as analysed by standard flow cytometry (Flow) or ImageStream (IS). LS (low scatter), HS (high scatter not including MaV) and MaV. (D) Chart of data in C, depicted as percentage change. This data demonstrates that a 1.2 μm filter removes the MaV population from SCD PFP and that it is this population that is detected by standard flow cytometry.

FIG. 8—Size estimation of 28 days cold storage-induced RCDP using the NansoSight LM10 system.

(A) Overlay of size distribution data of 4 individual measurements. The size distribution of the tracked particles of each measurement showed a close overlay indicating particle size lower than 300 nm. (B) Combined size distribution data profile of 4 individual measurements +/− the standard error of the mean. Peak position was displayed at 155 nm. (C) Sample description based on merged results. Mean particle size was established at 162.5 nm based on the results of 4 consecutive measurements. (D) Size-intensity scatter plots and size-concentration-intensity 3D plots representative to each measurement. (E) Single measurement details.

FIG. 9—ImageStream gating strategy

SCD plasma was processed and stained as described. (A) Scatter (channel 06) intensity was plotted against Scatter Max Pixel and gates were placed around speed beads, low scatter and high scatter events. Low scatter events (B) and high scatter events (C) were plotted in a histogram of ‘Brightfield Area’ and a gate placed on low area events and high area events respectively. Scatter plots of channel 02 vs channel 11 fluorescence intensity were created for low scatter & low area events.

### DETAILED DESCRIPTION

Various preferred features and embodiments of the present invention will now be described by way of non-limiting examples. This disclosure is not limited by the exemplary methods and materials disclosed herein, and any methods and materials similar or equivalent to those described herein can be used in the practice or testing of embodiments of this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

Numeric ranges are inclusive of the numbers defining the range.

### Red Cell-Derived Particles (RCDP)

In one aspect, the present invention provides a method of decreasing hypercoagulability and/or increasing plasma clotting time, comprising removing red cell-derived particles (RCDP). The method may be an ex vivo method.

The plasma may be isolated plasma. As used herein “isolated plasma” is plasma which has been isolated from a subject. The term “isolated plasma” does not encompass plasma which is present in a subject's body.

As used herein, “plasma clotting time” is the time it takes for the plasma to undergo coagulation. As used herein, “hypercoagulability” is when there is an increased tendency toward blood coagulation. As used herein, “coagulation” or “clotting” is the process by which blood changes from a liquid to a gel, forming a blood clot.

As used herein, “red cell-derived particles” (RCDP) can be erythrocyte or reticulocyte-derived micelles, vesicles and/or membrane fragments.

Erythrocytes are also known as red blood cells (RBCs), red cells, red blood corpuscles, haematids, or erythroid cells. Reticulocytes are immature erythrocytes. Erythrocyte and reticulocyte-derived particles are particles formed from erythrocyte and reticulocytes.

As used herein, “micelles” are closed structures formed by one or more lipid monolayers. As used herein, “vesicles” are closed structures formed by one or more lipid bilayers. The micelles and vesicles are typically spherical. As used herein, “membrane fragments” are open structures formed by one or more lipid bilayers. The lipids may be phospholipids.

The inventors have surprisingly found that there are different subpopulations of RCDP. In the methods of the present invention one or more of the different RCDP subpopulations can be removed. In some embodiments, only one RCDP subpopulation is removed.

During cell activation or eryptosis red cell-derived particles can bud from erythrocyte membranes. Sealed red cell-derived particles formed by this process are referred to herein as “right-side out red cell-derived particles”. Right-side out red cell-derived particles may include “erythrocyte vesicles”, e.g. “erythrocyte microvesicles” and “erythrocyte macrovesicles”. Unsealed red cell-derived particles may also be formed by the same process and are referred to herein as “erythrocyte-derived membrane fragments”.

During maturation, reticulocytes can release inside-out autophagic vesicles. Autophagic vesicles of reticulocyte or erythrocyte origin are referred to herein as “inside-out red cell-derived particles”.

The RCDP may comprise or consist of right-side out RCDP, erythrocyte-derived membrane fragments, and/or inside-out RCDP. In preferred embodiments the RCDP comprise or consist of right-side out RCDP and/or erythrocyte-derived membrane fragments. In more preferred embodiments the RCDP comprise or consist of right-side out RCDP.

The RCDP may comprise or consist of erythrocyte vesicles (including erythrocyte microvesicles and erythrocyte macrovesicles), erythrocyte-derived membrane fragments, and autophagic vesicles of reticulocyte or erythrocyte origin. In preferred embodiments the RCDP comprise or consist of erythrocyte vesicles and erythrocyte-derived membrane fragments. In more preferred embodiments the RCDP comprise or consist of erythrocyte vesicles (including erythrocyte microvesicles and erythrocyte macrovesicles). In even more preferred embodiments the RCDP comprise or consist of erythrocyte macrovesicles.

Preferably, in the methods of the present invention erythrocyte macrovesicles are removed. In such methods, other RCDP may also be removed, for example one or more of the other RCDP subpopulations may be removed. In some embodiments, only erythrocyte macrovesicles are removed.

### RCDP Surface Markers

The RCDP may have on their surface one or more markers present on the surface of erythrocytes or reticulocytes. Markers present on the surface of erythrocytes or reticulocytes will be well known to those of skill in the art.

### Blood Group Active Proteins

The RCDP may have on their surface one or more blood group active proteins. The RCDP may have on their surface all blood group active proteins.

The surface of the human red blood cell is dominated by a small number of abundant blood group active proteins (Anstee, D. J., 1990. Vox Sanguinis, 58(1), pp.1-20). Major blood group active proteins include anion exchanger 1 (AE1), Glycophorin A (GPA), glucose transporter 1 (GLUT1), Glycophorin B (GPB), Blood group Rh(D) polypeptide (RHD), Blood group Rh(CE) polypeptide (RHCE), Rh associated glycoprotein (RHAG), Glycophorin C (GPC) and Glycophorin D (GPD).

Other blood group active proteins include Aquaporin 1 (AQP1), Duffy glycoprotein (DARC), Kell Glycoprotein (KEL), Urea transporter (UT-B), Decay accelerating factor (CD55), Lutheran glycoprotein (LU), LW glycoprotein (ICAM-4), Kx glycoprotein (KX), ADP-ribosyltransferase (ART), Tetraspanin (CD151), Erythrocyte membrane-associated protein (ERMAP), CD44, Xg^{a} glycoprotein (XGA), CR1 (CD35), EMMPRIN (CD147), Acetylcholinesterase (AChE), Semaphorin 7A (SEMA7A), and Aquaporin 3 (AQP3) (Anstee, D. J., 2011. Vox sanguinis, 100(1), pp.140-149).

The RCDP may have on their surface one or more marker selected from the list consisting of: GPA, AE1, GLUT1, GPC, GPB, RHD, RHCE, RHAG, GPD, AQP1, DARC, KEL, UT-B, CD55, LU, ICAM-4, KX, ART, CD151, ERMAP, CD44, XGA, CD36, CD147, AChE, SEMA7A and AQP3. The RCDP may have on their surface GPA, AE1, GLUT1, GPC, GPB, RHD, RHCE, RHAG, GPD, AQP1, DARC, KEL, UT-B, CD55, LU, ICAM-4, KX, ART, CD151, ERMAP, CD44, XGA, CD36, CD147, AChE, SEMA7A and AQP3.

The RCDP may have on their surface one or more marker selected from the list consisting of: GPA, AE1, GLUT1, and GPC. The RCDP may have on their surface GPA, AE1, GLUT1, and GPC.

The RCDP may have on their surface Glycophorin A (GPA) e.g. UniProt entry P02724. GPA is also known as GYPA, CD235a, GPA, GPErik, GPSAT, HGpMiV, HGpMiXI, HGpSta(C), MN, MNS, PAS-2, glycophorin A (MNS blood group). GPA consists of 131 amino acids, which constitute three domains: (i) a heavily glycosylated N-terminal extracellular domain of 72 amino acids, (ii) a hydrophobic intramembranous domain of 23 amino acids, and (iii) a C-terminal cytoplasmic domain of 36 amino acids. GPA is the major intrinsic membrane protein of the erythrocyte. There are about one million copies of GPA protein per erythrocyte.

The RCDP may have on their surface Anion exchanger 1 (AE1) e.g. UniProt entry P02730. AE1 is also known as SLC4A1, solute carrier family 4 (anion exchanger), member 1 (Diego blood group), AE1, BND3, CD233, DI, EMPB3, EPB3, FR, RTA1A, SW, WD, WD1, WR, CHC, SAO, SPH4, solute carrier family 4 member 1 (Diego blood group). AE1 is present in the erythrocyte cell membrane. There are about one million copies of AE1 protein per erythrocyte.

The RCDP may have on their surface Glucose transporter 1 (GLUT1) e.g. UniProt entry P11166. GLUT1 is also known as SLC2A1, Solute carrier family 2, facilitated glucose transporter member 1, Glucose transporter type 1, erythrocyte/brain and epG2 glucose transporter. GLUT1 accounts for 2 percent of the protein in the plasma membrane of erythrocytes. There are about half a million copies of GLUT1 protein per erythrocyte.

The RCDP may have on their surface Glycophorin C (GPC) e.g. UniProt entry P04921. GPC is also known as GYPC, CD236/CD236R; glycoprotein beta, glycoconnectin, PAS-2′. GPC is a minor sialoglycoprotein in human erythrocyte membranes. GPC plays a functionally important role in maintaining erythrocyte shape and regulating membrane material properties. There are about 60,000-120,000 copies of GPC protein per erythrocyte.

The RCDP may have on their surface one or more markers present on the extracellular and/or intracellular surface of erythrocytes or reticulocytes. For example, the RCDP may have on their surface one or more markers from the extracellular and/or intracellular domains of membrane proteins present on the surface of erythrocytes or reticulocytes. Preferably, the RCDP have on their surface one or more markers present on the extracellular surface of erythrocytes or reticulocytes. The RCDP may have on their surface one or more markers selected from the extracellular domains of membrane proteins present on the surface of erythrocytes or reticulocytes.

Right-side out RCDP (e.g. erythrocyte vesicles) may have on their surface one or more markers present on the extracellular surface of erythrocytes or reticulocytes. RCDP may have on their surface the extracellular domain of one or more blood group active proteins, especially when the RCDP comprise or consist of right-side out RCDP. RCDP may have on their surface the extracellular domain of GPA, the extracellular domain of AE1, the extracellular domain of GLUT1, and/or the extracellular domain of GPC, especially when the RCDP comprise or consist of right-side out RCDP. Preferably the RCDP have on their surface the extracellular domain of GPA, especially when the RCDP comprise or consist of right-side out RCDP.

Inside-out RCDP (autophagic vesicles of reticulocyte or erythrocyte origin) may have on their surface one or more markers present on the intracellular surface of erythrocytes or reticulocytes. RCDP may have on their surface the intracellular (cytoplasmic) domain of one or more blood group active proteins, especially when the RCDP comprise or consist of inside-out RCDP. RCDP may have on their surface the intracellular domain of GPA, the intracellular domain of AE1, the intracellular domain of GLUT1, and/or the intracellular domain of GPC, especially when the RCDP comprise or consist of inside-out RCDP. Preferably the RCDP have on their surface the intracellular (cytoplasmic) domain of GPA, especially when the RCDP comprise or consist of inside-out RCDP.

Erythrocyte-derived membrane fragments may have on their surface one or more markers present on the extracellular and/or intracellular surface of erythrocytes or reticulocytes. RCDP may have on their surface the extracellular domain and/or intracellular (cytoplasmic) domain of one or more blood group active proteins, especially when the RCDP comprise or consist of erythrocyte-derived membrane fragments. RCDP may have on their surface the extracellular domain and/or intracellular domain of GPA, the extracellular domain and/or intracellular domain of AE1, the extracellular domain and/or intracellular domain of GLUT1, and/or the extracellular domain and/or intracellular domain of GPC, especially when the RCDP comprise or consist of erythrocyte-derived membrane fragments. Preferably the RCDP have on their surface the extracellular domain and/or intracellular (cytoplasmic) domain of GPA, especially when the RCDP comprise or consist of Erythrocyte-derived membrane fragments.

The presence of blood group active proteins on the surface of the RCDP may be determined by any suitable method known to those of skill in the art. For example, the presence of blood group active proteins on the surface of the RCDP may be determined by flow cytometry, optical microscopy, and/or electron microscopy. Blood group active proteins can be labelled by fluorescent probes (e.g. fluorescently-labelled antibodies) and detected by flow cytometry, including imaging flow cytometry, and/or optical microscopy. Blood group active proteins can be labelled by gold-conjugated probes (e.g. antibodies conjugated to gold) and detected by electron microscopy.

### Phosphatidylserine

The RCDP may have on their surface phosphatidylserine (PS).

Phosphatidylserine (abbreviated Ptd-L-Ser or PS) is a phospholipid and is a component of the cell membrane. PS is a glycerophospholipid consisting of two fatty acids attached in ester linkage to the first and second carbon of glycerol and serine attached through a phosphodiester linkage to the third carbon of the glycerol.

PS can promote thrombosis. When circulating platelets encounter the site of an injury, collagen and thrombin-mediated activation can cause externalization of PS from the inner membrane layer, where it serves as a pro-coagulant surface. This surface can act to orient coagulation proteases, specifically tissue factor (TF) and factor VII, facilitating further proteolysis, activation of factor X, and ultimately generating thrombin. RCDP may be pro-thrombotic and pro-inflammatory due to the surface expression of PS.

The presence of PS on the surface of the RCDP may be determined by any suitable method known to those of skill in the art. For example, the presence of PS on the surface of the RCDP may be determined by flow cytometry, including imaging flow cytometry, optical microscopy, and/or electron microscopy. PS can be labelled by fluorescent probes (e.g. fluorescent Annexin V) and detected by flow cytometry and/or optical microscopy.

The RCDP may have on their surface one or more blood group active proteins and phosphatidylserine (PS). The RCDP may have on their surface all blood group active proteins and phosphatidylserine (PS). Preferably, the RCDP have GPA and PS on their surface. More preferably, the RCDP have PS and the extracellular domain of GPA on their surface.

### RCDP Particle Size

The RCDP may have a particle diameter of from 0.05 μm to 20 μm. In some embodiments the RCDP may have a particle diameter of from 0.1 μm to 12 μm, or from 1 μm to 12 μm, or from 1.2 μm to 12 μm, or from 3 μm to 12 μm. In some embodiments, the RCDP may have a particle diameter of from 0.1 μm to 8 μm, or from 1 μm to 8 μm, or from 1.2 μm to 8 μm, or from 3 μm to 8 μm. In some embodiments, the RCDP may have a particle diameter of from 0.05 μm to 1 μm.

Erythrocyte vesicles may have a particle diameter of from 0.05 μm to 12 μm. In some embodiments erythrocyte vesicles may have a particle diameter of from 0.1 μm to 12 μm, or from 0.1 μm to 8 μm.

Erythrocyte macrovesicles may have a particle diameter of greater than 1 μm and/or 12 μm or less (e.g. 11 μm or less, 10 μm or less, 9 μm or less, or 8 μm or less). In some embodiments erythrocyte macrovesicles have a particle diameter from 1.2 to 12 μm, or from 3 μm to 12 μm, or from 1.2 μm to 8 μm, or from 3 μm to 8 μm.

Erythrocyte microvesicles may have a particle diameter of from 0.05 μm to 1 μm. In some embodiments erythrocyte microvesicles have a particle diameter of 0.05 μm to 1.2 μm, or 0.1 μm to 1.2 μm, or 0.1 μm to 1 μm.

Erythrocyte-derived membrane fragments may have a particle diameter of from 0.05 μm to 6 μm. In some embodiments erythrocyte-derived membrane fragments have a particle diameter of 0.05 μm to 1 μm, or 0.05 μm to 1.2 μm, or 0.1 μm to 1.2 μm, or 0.1 μm to 1 μm.

Autophagic vesicles of reticulocyte or erythrocyte origin may have a particle diameter of from 0.05 μm to 2 μm. In some embodiments autophagic vesicles of reticulocyte or erythrocyte origin have a particle diameter of 0.05 μm to 1 μm, or 0.05 μm to 1.4 μm, or 0.1 μm to 1 μm, or 0.1 μm to 1.4 μm.

The RCDP may have a median particle diameter of from 4 μm to 8 μm, or from 6 μm to 9 μm, preferably from 6 μm to 8 μm, more preferably about 7 μm, especially when the RCDP consist of erythrocyte macrovesicles. Erythrocyte macrovesicles may have a median particle diameter of from 4 μm to 8 μm, or from 6 μm to 9 μm, preferably from 6 μm to 8 μm, more preferably about 7 μm.

As used herein, the “particle diameter” is the distance of the longest dimension of the particle. This is also known as dₘₐₓ, or the diameter of an equivalent sphere with the same maximum length as the particle. The “median particle diameter” is the value of the particle diameter which divides the particle population exactly into two equal halves i.e. there is 50% of the distribution above this value and 50% below.

The particle diameter and median particle diameter can be determined by any method known to those of skill in the art, for example optical microscopy (e.g. spinning-disk confocal microscopy), electron microscopy, imaging flow cytometry, and/or nanoparticle tracking analysis. Suitable methods are described in Example 5.

Suitably, the particle diameter is determined by electron microscopy, for example transmission electron microscopy, especially when the RCDP comprise one or more of erythrocyte microvesicles, erythrocyte-derived membrane fragments, and autophagic vesicles of reticulocyte or erythrocyte origin.

Suitably, when using transmission electron microscopy, RCDP may be placed on a carbon coated copper grid, left to dry, then fixed in 4% (v/v) paraformaldehyde with 0.05% (v/v) glutaraldehyde at room temperature for 30 minutes. Grids may be counterstained with a solution of 0.3% (w/v) uranyl acetate in 1.8% methylcellulose for 10 minutes on ice then air-dried using the wire loop method. Grids may be examined on a transmission electron microscope (e.g. Tecnai12 120 kV BioTwin Spirit transmission electron microscope) and may be visualised using a camera (e.g. FEI CETA camera) and software (e.g. TIA software).

Suitably, the median particle diameter is determined by optical microscopy, for example confocal microscopy (e.g. spinning-disk confocal microscopy), especially when the RCDP consist of erythrocyte macrovesicles. In one embodiment, the median particle diameter of erythrocyte macrovesicles is determined by optical microscopy.

Suitably, when using confocal microscopy, RCDP may be placed on poly-L-lysine coated microscopy plater. RCDP may be examined using a spinning-disk confocal microscope (e.g. an Opera LX HCS spinning-disk confocal microscope) with a 60× (NA 1.2) water-immersion lens and analysed using software (e.g. Acapella software). The size and shape of RCDP may be determined using a circular Hough transform in XY axes to detect particles. The Hough transform tool is selective for objects with a high degree of radial symmetry and will ignore ellipses. An automated script may be used to determine the diameter.

The RCDP may have a particle diameter of from 0.05 μm to 12 μm and have on their surface one or more blood group active proteins and/or phosphatidylserine (PS).

The RCDP may have a median particle diameter of from 4 μm to 8 μm, preferably from 6 μm to 8 μm and have on their surface one or more blood group active proteins and phosphatidylserine (PS), especially when the RCDP consist of erythrocyte macrovesicles. Preferably, the RCDP have on their surface the extracellular domain of GPA, the extracellular domain of AE1, the extracellular domain of GLUT1, and/or the extracellular domain of GPC. More preferably, the RCDP have on their surface the extracellular domain of GPA.

### Methods for Removing RCDP

RCDP may be removed in the methods of the present invention by any suitable method.

Suitably, RCDP may be removed by affinity chromatography and/or size exclusion chromatography (i.e. filtration).

In some embodiments, the method of the present invention does not comprise solvent detergent treatment. As used herein “solvent detergent treatment” is a method used to inactivate enveloped viruses in plasma protein preparations and is described in e.g. Hellstern P, Solheim B G. Transfus Med Hemother. 2011;38(1):65-70.

### Affinity Chromatography

RCDP may be removed in the methods of the present invention by affinity chromatography.

As used herein, “affinity chromatography” is a method of separating biochemical mixture based on a highly specific interaction between molecules. Affinity chromatography exploits the differences in interaction strengths between the different molecules within a mobile phase and a stationary phase. The stationary phase (e.g. comprising a molecule which specifically binds a RCDP marker) may be loaded into a column. Then, the mobile phase (e.g. plasma containing RCDP) may be added to the stationary phase and the two phases allowed time to bind. The target biomolecules (e.g. RCDP markers) have a much higher affinity for the stationary phase, and may bind to the stationary phase. The amount of unwanted substance (e.g. RCDP) may be reduced in the mobile phase which can be eluted (e.g. modified plasma with reduced RCDP).

Suitably RCDP may be removed by a molecule which binds, suitably specifically binds, one or more markers expressed on the surface of RCDP (RCDP markers).

Molecules that specifically bind to RCDP markers can be prepared using methods well known by those of skill in the art.

The molecule used in the present invention may specifically bind to a RCDP marker selected from the list consisting of: GPA, AE1, GLUT1, GPC, PS, GPB, RHD, RHCE, RHAG, GPD, AQP1, DARC, KEL, UT-B, CD55, LU, ICAM-4, KX, ART, CD151, ERMAP, CD44, XGA, CD36, CD147, AChE, SEMA7A and AQP3.

The molecule used in the present invention may specifically bind to a RCDP marker selected from the list consisting of: GPA, AE1, GLUT1, GPC, and PS.

The molecule used in the present invention may specifically bind to GPA.

The molecule used in the present invention may specifically bind to AE1.

The molecule used in the present invention may specifically bind to GLUT1.

The molecule used in the present invention may specifically bind to GPC.

The molecule used in the present invention may specifically bind to PS.

The molecule used in the present invention may specifically bind to a marker present on the extracellular surface of erythrocytes or reticulocytes (i.e. a right-side out RCDP marker). The molecule used in f the present invention may specifically bind to the extracellular domain of a blood group active protein. The molecule used in the present invention may specifically bind to the extracellular domain of GPA, the extracellular domain of AE1, the extracellular domain of GLUT1, or the extracellular domain of GPC. Preferably the molecule used in the present invention may specifically bind to the extracellular domain of GPA.

The molecule used in the present invention may specifically bind to a marker present on the intracellular surface of erythrocytes or reticulocytes (i.e. an inside-out RCDP marker). The molecule used in the present invention may specifically bind to the intracellular domain of a blood group active protein. The molecule used in the present invention may specifically bind to the intracellular domain of GPA, the intracellular domain of AE1, the intracellular domain of GLUT1, or the intracellular domain of GPC.

The molecule used in the present invention may selectively or specifically bind to one or more RCDP markers, and thus may have a greater binding affinity for one or more RCDP markers, as compared to its binding affinity for markers for other blood or plasma components. Suitably, “specifically binds” as used herein means that the molecule does not bind to the other markers or binds with a greatly reduced affinity compared to the binding to the RCDP marker to which it specifically binds (e.g. with an affinity of at least 10, 50, 100, 500, 1000 or 10000 times less than its affinity for the RCDP marker to which it specifically binds). Thus, the molecule as referred to herein may bind to its RCDP marker with at least 10, 50, 100, 500, 1000 or 10000 times the affinity of its binding to the other markers. The binding affinity of the molecule can be determined using methods well known in the art such as with the Biacore system.

The molecule used in the present invention may be any molecule capable of binding an RCDP marker. In particular, the molecule used in the present invention may be any protein or peptide that possesses the ability to specifically bind to an RCDP marker. The molecule may be any naturally occurring, synthetic, semi-synthetic, or recombinantly produced binding partner for RCDP marker. Illustrative molecules include antibodies, antibody fragments or derivatives, extracellular domains of receptors, and ligands for cell surface molecules/receptors.

The molecule may be an antibody (Ab) or may be derived from an antibody. An antibody-derived molecule can be a fragment of an antibody or a genetically engineered product of one of more fragments of the antibody, which fragment is involved in binding with the antigen. Examples include a camelid antibody (VHH), an antigen-binding fragment (Fab), a variable region (Fv), a single chain antibody (scFv), a single-domain antibody (sdAb), a heavy chain variable region (VH), a light chain variable region (VL), and a complementarity determining region (CDR). Preferably, the molecule is an antibody.

An antibody recognises an antigen via the fragment antigen-binding (Fab) variable region. Antibodies are glycoproteins belonging to the immunoglobulin superfamily. They constitute most of the gamma globulin fraction of the blood proteins. They are typically made of basic structural units, each with two large heavy chains and two small light chains. Camelid antibodies (VHH) lack light chains, and consist of two heavy chains attached to variable domains.

“Antigen-binding fragment” (Fab) refers to a region on an antibody that binds to antigens. It is composed of one constant and one variable region of each of the heavy and the light chain.

“Fv” refers to the smallest fragment of an antibody to bear the complete antigen binding site. An Fv fragment consists of the variable regions of a single light chain bound to the variable region of a single heavy chain. “Single chain antibody” (scFv) refers to an engineered antibody consisting of a light chain variable region and a heavy chain variable region connected to one another directly or via a peptide linker sequence. The peptide linker sequence is usually about 10 to 25 amino acids in length, rich in glycine for flexibility, and serine or threonine for solubility. The peptide linker sequence can either connect the N-terminus of the heavy chain variable region with the C-terminus of the light chain variable region, or vice versa.

“Single-domain antibody” (sdAb), also known as a nanobody, refers to an antibody fragment consisting of a single monomeric variable antibody domain. Accordingly, a sdAb may be a heavy chain variable region (VH) or a light chain variable region (VL).

“Heavy chain variable region” or “VH” refers to the fragment of the heavy chain of an antibody that contains three CDRs interposed between flanking stretches known as framework regions, which are more highly conserved than the CDRs and form a scaffold to support the CDRs. “Light chain variable region” or “VL” refers to the fragment of the light chain of an antibody that contains three CDRs interposed between framework regions.

“Complementarity determining region” or “CDR” with regard to antibody or antigen-binding fragment thereof refers to a highly variable loop in the variable region of the heavy chain of the light chain of an antibody. CDRs can interact with the antigen conformation and largely determine binding to the antigen (although some framework regions are known to be involved in binding). The heavy chain variable region and the light chain variable region each contain 3 CDRs (heavy chain CDRs 1, 2 and 3 and light chain CDRs 1, 2 and 3, numbered from the amino to the carboxy terminus).

Antibodies, and derivatives and fragments thereof that specifically bind to RCDP markers can be prepared using methods well known by those of skill in the art. Such methods include phage display, methods to generate human or humanized antibodies, or methods using transgenic animal or plant engineered to produce human antibodies. Phage display libraries of partially or fully synthetic antibodies are available and can be screened for an antibody or fragment thereof that can bind to the target molecule. Phage display libraries of human antibodies are also available. Once identified, the amino acid sequence or polynucleotide sequence encoding for the antibody (or derivative or fragment thereof) can be isolated and/or determined. The sequence of the antibody can be used to design suitable derivatives or fragments thereof.

Examples of molecules that can be used in the invention are further described below.

BRIC163 is an antibody which specifically binds the intracellular (cytoplasmic) domain of GPA (Okubo Y, et al. Vox Sang. 1988;54(2):107-111). In one embodiment, the molecule used in the present invention is BRIC163.

BRIC256 and R10 are antibodies which specifically binds the extracellular domain of GPA. (Mankelow T J, et al. Blood. 2015;126(15):1831-1834; Reid M E, et al. Transfusion clinique et biologique: journal de la Societe francaise de transfusion sanguine. 1997;4(1):57-64). In one embodiment, the molecule used in the present invention is BRIC256 or R10.

BRIC132, BRIC155 and BRAC66 are antibodies which specifically bind the intracellular domain of AE1 (van Tits L J, et al. Biochem Biophys Res Commun. 2009;390(1):161-164; Kasar M, et al. J Thromb Thrombolysis. 2014;38(2):167-175). In one embodiment, the molecule used in the present invention is BRIC132, BRIC155 or BRAC66.

Annexin V is a protein which specifically binds PS. In one embodiment, the molecule used in the present invention is Annexin V.

Preferably, the molecule is immobilised. As used herein an “immobilised molecule” is one whose movement in space has been restricted either completely or to a small limited region by attachment to a solid structure. Suitably, the molecule may be immobilised by adsorption, covalent binding, entrapment, encapsulation and/or crosslinking. Suitably the molecule may be immobilised on a substrate and/or matrix. Suitable substrates and matrices will be well known to those of skill in the art (see e.g. Elnashar, M. M., 2010. Journal of Biomaterials and Nanobiotechnology, 1(1), pp.61-76). For example the substrate or matrix may be one or more of: a polysaccharide (e.g. cellulose, dextran, starch, agar, agarose, alginate, carrageenans, chitin, and chitosan); a protein (e.g. collagen, gelatin, albumin, and ferritin); a synthetic polymer (e.g. polystyrene, polyacrylate, polymethacrylate, polyacrylamides, hydroxyalkyl methacrylate, vinyl polymer, maleic anhydride polymer, polyethyleneglycol, and aldehyde-based polymer); a mineral (e.g. attapulgite clays, bentonite, kieselgur, pumic stone, hornblend, diatomaceous earth, and sand); and a fabricated material (e.g. non-porous glass, controlled pore glass, controlled pore metal oxides, alumina catalyst, porous silica, silochrome, iron oxide, and stainless steel).

Suitably the molecule may be immobilised on the stationary phase of an affinity column.

Suitably the molecule may be immobilised on a bead, for example a magnetic bead.

### Affinity Column

RCDP may be removed in the methods of the present invention by an affinity column which binds, suitably specifically binds, one or more markers expressed on the surface of RCDP (RCDP markers).

As used herein, an “affinity column” is a device used for affinity chromatography.

The affinity column of the invention may comprise a molecule of the invention which binds, suitably specifically binds, one or more RCDP markers, as described herein. The molecule may be immobilised on the stationary phase of the affinity column.

The molecule which specifically binds a RCDP marker can be immobilised on the stationary phase of the affinity column by covalent bonds.

Suitable stationary phases will be well known to those of skill in the art. Suitably, the stationary phase may be insoluble in plasma. Suitably, the stationary phase may be a solid. Suitably, the stationary phase may be a resin or a gel, for example a resin or gel bead. Suitably, the stationary phase may be agarose, sepharose, cellulose, dextran, polyacrylamide, latex or controlled pore glass. The stationary phase may be agarose, for example beaded agarose (e.g. crosslinked 4%, 5% or 6% beaded agarose). The stationary phase may be polyacrylamide, for example beaded polyacrylamide.

The affinity column may be an immunoaffinity column, i.e. an affinity column in which an antibody which specifically binds an RCDP marker is immobilised on the stationary phase.

Suitable methods for preparing an affinity column of the invention will be well known to those of skill in the art.

Suitably, the molecule used in the invention (e.g. antibody) can be immobilised on the stationary phase (e.g. agarose) by amine coupling. Aldehyde groups react with primary amines to form Schiff bases. The Schiff base is then reduced with a suitable reducing agent (e.g. sodium cyanoborohydride) to form a covalent bond and couple the protein to the stationary phase.

Suitably, the molecule used in the invention (e.g. antibody) can be immobilised on the stationary phase (e.g. agarose) by NHS coupling. NHS groups react to primary amines forming a covalent bond. The reaction is performed at near neutral pH.

Suitably, the molecule used in the invention (e.g. antibody) can be immobilised on the stationary phase (e.g. agarose) by sulfhydryl coupling. lodoacetyl groups can be used to covalently attach proteins to the stationary phase through free sulfhydryls. In the case of antibodies, a mild reducing agent (2-mercaptoethylamine) can be used to generate free sulfhydryls in the constant domain, without destroying antigen binding affinities.

Suitably, the molecule used in the invention (e.g. antibody) can be immobilised on the stationary phase (e.g. agarose) by coupling to Immobilized Protein A, Immobilized Protein G or Immobilized Protein A/G. These bind the antibody constant domain ensuring the antigen binding domain are facing away from the stationary phase for optimal binding. The selection is dependent of the affinity of the protein A, G or A/G for the antibody. A crosslinker can be used, such as DSS.

The affinity column may be suitable for use in an apheresis device.

### Affinity Beads

RCDP may be removed in the methods of the present invention by a bead which binds, suitably specifically binds, one or more markers expressed on the surface of RCDP (RCDP markers).

The bead of the invention may comprise a molecule which binds, suitably specifically binds, one or more RCDP markers, as described herein. The molecule may be immobilised on the bead.

Suitable beads will be well known to those of skill in the art. Suitably, the bead may be insoluble in plasma.

In some embodiments, the bead is a resin or gel bead (i.e. porous). Suitably, the bead may be an agarose bead, a sepharose bead, a cellulose bead, a dextran bead, a polyacrylamide bead, a latex bead or a controlled pore glass bead. The bead may be an agarose bead (e.g. crosslinked 4%, 5% or 6% agarose). The bead may be a polyacrylamide bead. A resin or gel bead may be about 50-150 μm in diameter.

Suitable methods for preparing resin or gel bead will be well known to those of skill in the art.

Suitably, the molecule used in the invention (e.g. antibody) can be immobilised on the resin or gel bead (e.g. agarose) by sulfhydryl coupling, amine coupling, NHS coupling, or by coupling to Immobilized Protein A, Immobilized Protein G or Immobilized Protein A/G.

The beads may be used in an affinity column. For example, the bead may be used as the stationary phase of an affinity column. A beaded format allows resins to be supplied as wet slurries that can be easily dispensed to fill and “pack” columns with resin beds of any size.

In some embodiments, the bead is a magnetic bead. Magnetic bead may be about 1-4 μm in diameter. Suitably, the bead may be solid (i.e. non-porous). Suitably, the magnetic bead may be a superparamagnetic iron oxide bead. The superparamagnetic iron oxide bead may be covalently coated with silane derivatives. Suitably, the molecule used in the invention (e.g. antibody) can be immobilised on the magnetic bead by coupling to the silane derivatives.

Suitably, affinity purification with magnetic particles is not performed in-column. A few microliters of magnetic beads can be mixed with several hundred microliters of sample as loose slurry. During mixing, the beads remain suspended in the sample solution, allowing affinity interactions to occur with the RCDP. After sufficient time for binding has been given, the beads are collected and separated from the sample using a powerful magnet. Typically, simple bench-top procedures are done in micro-centrifuge tubes, and pipetting or decanting is used to remove the sample (or wash solutions, etc.) while the magnetic beads are held in place at the bottom or side of the tube with a suitable magnet.

The bead may be suitable for use in an apheresis device.

### Apheresis Kit or Device

RCDP may be removed in the methods of the present invention by an apheresis device or kit, which comprises a molecule, an affinity column, and/or a bead of the present invention.

Preferably the apheresis device or kit of the present invention comprises an affinity column which binds, suitably specifically binds, one or more markers expressed on the surface of RCDP (RCDP markers). The affinity column may be the affinity column of the invention, as described herein.

As used herein, an “apheresis device” is a device which receives blood removed from a patient or donor's body and separates it into its various components: plasma, platelets, white blood cells and red blood cells. One component can be separated and optionally processed and the remainder of the blood returned to the donor's circulation. An “apheresis kit” is a kit of apheresis device parts.

The apheresis device may be a plasmapheresis device. The apheresis kit may be a plasmapheresis kit. Plasmapheresis is the removal, treatment, and return or exchange of blood plasma or components thereof from and to the blood circulation.

The apheresis device or kit may be used for extracorporeal therapy. The apheresis device or kit may be used for extracorporeal immunoadsorption.

### Size Exclusion Chromatography

RCDP may be removed in the methods of the present invention by size exclusion chromatography.

As used herein, “size-exclusion chromatography” is a chromatographic method in which molecules in solution are separated by their size, and in some cases molecular weight. Size-exclusion chromatography encompasses filtration and gel filtration.

In the methods of the present invention RCDP can be selectively removed from plasma based on their size, as described herein. A filter with a pore size smaller than the particle diameter of the RCDP can be used to selectively remove RCDP. Pore size relates to the filter's ability to filter out particles of a certain size. For example, a 0.2 micron (μm) membrane will filter out particles with a diameter of 0.2 microns or larger from a filtration stream.

Filters with different pore sizes are commercially available. Pore size may be determined by any suitable technique known to those of skill in the art. Suitably, pore size may be determined by scanning electron microscopy, where a small section of membrane is appropriately treated, put in the microscope, and evaluated, using appropriate imaging software. Suitably pore size may be determined by porosimetry, a physical method where liquid is forced into the membrane under pressure and the penetration profile is analyzed mathematically to determine pore size. Suitably, pore size may be determined by particle challenge uses particles of defined size to determine the minimum size that can be retained by the filter.

RCDP may be selectively removed by a filter having a pore size of 0.03 μm or less or 0.05 μm or less, suitably a pore size of about 0.03 μm or a pore size of about 0.05 μm. A portion of RCDP may be selectively removed by a filter having a pore size of 0.1 μm or less, 0.2 μm or less, 0.4 μm or less, 0.45 μm or less, 0.5 μm or less, 0.65 μm or less, 0.8 μm or less, 1 μm or less, 1.2 μm or less, 1.5 μm or less, 2 μm or less, 3 μm or less, 4 μm or less, or 5 μm or less. Suitably, the filter may have a pore size of greater than 0.03 μm or greater than 0.05 μm. A portion of RCDP may be selectively removed by a filter having a pore size of about 0.1 μm, about 0.2 μm, about 0.4 μm, about 0.45 μm, about 0.65 μm, about 0.8 μm, about 1 μm, about 1.2 μm, about 2 μm, about 3 μm, or about 5 μm.

Erythrocyte macrovesicles may be selectively removed by a filter having a pore size of 0.05 μm or less, 0.1 μm or less, 0.2 μm or less, 0.4 μm or less, 0.45 μm or less, 0.5 μm or less, 0.65 μm or less, 0.8 μm or less, 1 μm or less, 1.2 μm or less, 1.5 μm or less, 2 μm or less, or 3 μm or less. Suitably, the filter may have a pore size of greater than 0.03 μm or greater than 0.05 μm.

Erythrocyte macrovesicles may be selectively removed by a filter having a pore size of 0.1-2 μm, 0.5-2 μm, 0.8-1.6 μm, or 1.0-1.5 μm. Erythrocyte macrovesicles may be selectively removed by a filter having a pore size of about 0.1 μm, about 0.2 μm, about 0.4 μm, about 0.45 μm, about 0.65 μm, about 0.8 μm, about 1 μm, about 1.2 μm, about 2 μm, about 3 μm, or about 5 μm. Erythrocyte macrovesicles may be selectively removed by a filter having a pore size of about 1 μm, about 1.2 μm, or about 2 μm, preferably about 1.2 μm.

Size exclusion chromatography (e.g. filtration) can be performed by any suitable method.

Suitably, RCDP may be removed in the methods of the present invention by syringe drive filtration. Suitably the filter may be a polyethersulfone (PES) membrane, a polyester (PETE) hydrophilic membrane, a cellulose acetate membrane, a mixed cellulose ester membrane (MCE), or a polyacrylonitrile (PAN) membrane.

Suitably, RCDP may be removed in the methods of the present invention by a filtration column, e.g. a gel filtration column. Suitably, the chromatography column may be packed with fine, porous beads which are composed of dextran polymers (e.g. Sephadex), agarose (e.g. Sepharose), or polyacrylamide (e.g. Sephacryl or BioGel P).

RCDP may be removed in the methods of the present invention by an apheresis device or kit, which comprises a filtration column, as described herein.

The apheresis device or kit of the present invention can comprise a filtration column. The filtration column may be the filtration column of the invention, as described herein.

### Plasma and Modified Plasma

### Plasma

Blood plasma may be isolated from a subject (as described herein), prior to being modified according to the present invention.

Blood plasma (herein referred to as “plasma”) is the fluid of whole blood that consists of water and its dissolved constituents including especially proteins (such as albumin, fibrinogen, and globulins). Plasma is the liquid part of the blood that carries cells and proteins throughout the body. Plasma makes up about 55% of the body's total blood volume. Plasma has a density of approximately 1.025 g/ml.

Suitably, the plasma to be modified the present invention may be fresh plasma. Suitably, the plasma to be modified the present invention may have been stored prior to use of the method of the invention. The plasma to be modified the present invention may be fresh frozen plasma.

In some embodiments, the plasma has not been solvent detergent treated. In some embodiments, the plasma is not solvent detergent plasma. As used herein “solvent detergent plasma” is a form of blood plasma made from plasma which is processed by solvent detergent treatment.

Plasma may be provided by any suitable method known to those of skill in the art. Suitably, plasma may be separated from isolated blood by spinning a tube of fresh blood in a centrifuge until the blood cells fall to the bottom of the tube. The plasma may then poured or drawn off.

Suitably, the plasma to be modified in the present invention may be platelet-free plasma. Plasma may be separated from blood by any suitable method known to those of skill in the art. Suitably, platelets may be separated from isolated plasma by spinning a tube of plasma in a centrifuge until the platelets fall to the bottom of the tube. The plasma may then poured or drawn off.

The plasma to be modified in the present invention may have a volume of about 50 ml or greater, about 100 ml or greater, about 250 ml or greater, about 500 ml or greater, about 1 litre or greater, about 2 litres or greater, or about 3.5 litres or greater. Suitably, the plasma to be modified in the present invention may have a volume of about 3.5 litres.

Suitably, the plasma to be modified may be a portion of the subject's total plasma, for example about 5% of the subject's total plasma or greater, about of the subject's total plasma 10% or greater, about of the subject's total plasma 20% or greater, about 30% of the subject's total plasma or greater, about 40% of the subject's total plasma or greater, about 50% of the subject's total plasma or greater, about 60% of the subject's total plasma or greater, about 70% of the subject's total plasma or greater, about 80% of the subject's total plasma or greater, about 90% of the subject's total plasma or greater, or about 100% of the subject's total plasma. Preferably, RCDP are removed from substantially all the subject's plasma.

In the method of the invention, suitably 10% or more, 20% or more, 30% or more, 40% or more, 50% or more, 60% or more, 70% or more, 80% or more, 90% or more, or about 100% of the total RCDP are removed from the plasma. Preferably, about 90% or more of the total RCDP are removed from the plasma.

In the method of the invention, suitably 10% or more, 20% or more, 30% or more, 40% or more, 50% or more, 60% or more, 70% or more, 80% or more, 90% or more, or about 100% of any of the RCDP subpopulations (as defined herein) are removed from the plasma. Preferably, about 90% or more of any of the RCDP subpopulations (as defined herein) are removed from the plasma.

In the method of the invention, suitably 10% or more, 20% or more, 30% or more, 40% or more, 50% or more, 60% or more, 70% or more, 80% or more, 90% or more, or about 100% of the erythrocyte vesicles and/or erythrocyte-derived membrane fragments are removed from the plasma. Suitably, about 90% or more of the erythrocyte vesicles and/or erythrocyte-derived membrane fragments are removed from the plasma.

In the method of the invention, preferably 10% or more, 20% or more, 30% or more, 40% or more, 50% or more, 60% or more, 70% or more, 80% or more, 90% or more, or about 100% of the erythrocyte macrovesicles are removed from the plasma. More preferably, about 90% or more of the erythrocyte macrovesicles are removed from the plasma.

### Modified Plasma

In one aspect, the present invention provides plasma obtained or obtainable by a method of the present invention.

Plasma in which RCDP has been removed is referred to herein as “modified plasma”. This may also be called “low-RCDP plasma”. “Un-modified plasma” refers to plasma in which RCDP has not been removed, i.e. plasma isolated from the subject which is not processed with the method of the invention.

Suitably, the modified plasma may be fresh modified plasma. Suitably, the modified plasma may be frozen.

In some embodiments, the modified plasma has not been solvent detergent treated. In some embodiments, the modified plasma is not solvent detergent plasma.

Suitably the modified plasma of the present invention has 90% or less, 80% or less, 70% or less, 60% or less, 50% or less, 40% or less, 30% or less, 20% or less, 10% or less total RCDP compared to un-modified plasma. Preferably, the modified plasma 10% or less total RCDP compared to un-modified plasma.

Suitably the modified plasma of the present invention has 90% or less, 80% or less, 70% or less, 60% or less, 50% or less, 40% or less, 30% or less, 20% or less, 10% or less of any of the RCDP subpopulations (as defined herein) compared to un-modified plasma. Preferably, the modified plasma 10% or less of any of the RCDP subpopulations (as defined herein) compared to un-modified plasma.

Suitably the modified plasma of the present invention has 90% or less, 80% or less, 70% or less, 60% or less, 50% or less, 40% or less, 30% or less, 20% or less, 10% or less erythrocyte vesicles and/or erythrocyte-derived membrane fragments compared to un-modified plasma. Preferably, the modified plasma 10% or less erythrocyte vesicles and/or erythrocyte-derived membrane fragments compared to un-modified plasma.

Suitably the modified plasma of the present invention has 90% or less, 80% or less, 70% or less, 60% or less, 50% or less, 40% or less, 30% or less, 20% or less, 10% or less erythrocyte macrovesicles compared to un-modified plasma. Preferably, the modified plasma 10% or less erythrocyte macrovesicles compared to un-modified plasma.

Suitably, the modified plasma of the present invention is substantially free or free of RCDP.

Suitably, the modified plasma of the present invention is substantially free or free of one or more of erythrocyte vesicles (including erythrocyte microvesicles and erythrocyte macrovesicles), erythrocyte-derived membrane fragments, and autophagic vesicles of reticulocyte or erythrocyte origin. Suitably, the modified plasma of the present invention is substantially free or free of erythrocyte vesicles and erythrocyte-derived membrane fragments. Preferably, the modified plasma of the present invention is substantially free or free of erythrocyte macrovesicles.

The amount of RCDP in plasma may be determined by any suitable method known to those of skill in the art. For example, flow cytometry, such as imaging flow cytometry, optical microscopy, such as confocal microscopy, and/or electron microscopy, such as transmission election microscopy. Preferably, the amount of RCDP in plasma is determined by flow cytometry, such as imaging flow cytometry.

### Plasma Clotting Time

The plasma clotting time may be increased by the methods of the present invention by at least two-fold, at least three-fold, or at least five-fold.

Suitably, the modified plasma has a clotting time at least two times longer than the un-modified plasma, preferably at least three times longer than the un-modified plasma, most preferably at least five times longer than the un-modified plasma.

Plasma clotting time may be determined by any suitable method known to those of skill in the art. Plasma clotting time may be the Factor Xa-activated clotting time, the thrombin time, the activated partial thromboplastin time, the prothrombin time, or the activated clotting time. Preferably plasma clotting time is the Factor Xa-activated plasma clotting time.

Suitably, the modified plasma has a clotting time which falls within normal ranges. Suitably a normal range may be a clotting time for control plasma. The control plasma may be isolated from a subject without a haemoglobinopathy, haemolytic anaemia or an erythrocyte membrane disorder. Suitably, the control plasma may be isolated from a subject without a sickle cell disease. The control plasma may be un-modified control plasma.

Suitably, the modified plasma has a clotting time which is approximately the same as the clotting time for control plasma. Suitably, the modified plasma may have a clotting time which is within 50% of the clotting time of the control plasma, within 40% of the clotting time of the control plasma, within 30% of the clotting time of the control plasma, within 20% of the clotting time of the control plasma, or within 10% of the clotting time of the control plasma.

Suitably, the modified plasma has a factor Xa-activated clotting time of 180 seconds or less. Suitably, the modified plasma has a factor Xa-activated clotting time of 60 seconds or more. Suitably, the modified plasma has a factor Xa-activated clotting time of about 60 to 180 seconds, or about 80 to 180 seconds, or about 90 to 160 seconds, or about 100 to 160 seconds.

Suitably, the modified plasma has a thrombin time of 22 seconds or less. Suitably, the modified plasma has a thrombin time of 8 seconds or more. Suitably, the modified plasma has a prothrombin time of about 8 to 22 seconds, or about 10 to 20 seconds, or about 12 to 18 seconds, or about 14 to 16 seconds.

Suitably, the modified plasma has an activated partial thromboplastin time of 80 seconds or less. Suitably, the modified plasma has an activated partial thromboplastin time of 15 seconds or more. Suitably, the modified plasma has an activated partial thromboplastin time of about 15 to 80 seconds, or about 20 to 70 seconds, or about 25 to 60 seconds, or about 30 to 50 seconds.

Suitably, the modified plasma has a prothrombin time of 15 seconds or less. Suitably, the modified plasma has a prothrombin time of 5 seconds or more. Suitably, the modified plasma has a prothrombin time of about 7 to 15 seconds, or about 9 to 15 seconds, or about 12 to 13 seconds.

Suitably, the modified plasma has an international normalized ratio of 1.5 or less. Suitably, the modified plasma has an international normalized ratio of 0.5 or more. Suitably, the modified plasma has an international normalized ratio of about 0.5 to 1.5, or about 0.6 to 1.4, or about 0.7 to 1.3, or about 0.8 to 1.2.

### Methods for Treating Disease

The present invention provides methods of treating diseases associated with reduced plasma clotting time and/or increased hypercoagulability.

In one aspect, the present invention provides a method of treating a disease, wherein the method comprises removing RCDP from plasma. The plasma may have been isolated from a subject.

In another aspect, the present invention provides a method of treating a disease in a subject, wherein the method comprises:
(i) isolating plasma from the subject;
(ii) removing RCDP from the plasma to provide modified plasma; and
(iii) administering the modified plasma to the subject.

The plasma may be isolated by any suitable method, for example those described herein.

RCDP may be removed by any suitable method, for example those described herein.

The modified plasma may administered by any suitable method. The modified plasma may be administered intravenously, alone or with other blood components (e.g. with one or more of red blood cells, white blood cells, and platelets).

### Apheresis and Plasmapheresis

The method of the invention may be an extracorporeal treatment.

The method of the invention may be performed as part of an apheresis procedure.

In one aspect, the present invention provides an apheresis procedure comprising removing RCDP. The apheresis device or kit of the present invention may be used.

Apheresis is an extracorporeal medical procedure that involves removing whole blood from a donor or patient and separating the blood into individual components so that one particular component can be removed. The remaining blood components are then re-introduced back into the bloodstream of the patient or donor. Preferably, the apheresis is autologous apheresis.

In some embodiments, the apheresis procedure comprises plasmapheresis and erythrocytapheresis. In some embodiments, the apheresis procedure comprises or consists of plasmapheresis, preferably autologous plasmapheresis.

In one aspect, the present invention provides a plasmapheresis procedure comprising removing RCDP. The plasmapheresis device or kit of the present invention may be used.

Plasmapheresis is an apheresis procedure that involves the removal, treatment, and return or exchange of blood plasma or components thereof from and to the blood circulation.

Erythrocytapheresis is an apheresis procedure by which erythrocytes (red blood cells) are separated from whole blood. Whole blood is extracted from a donor or patient, the red blood cells are separated, and the remaining blood is returned to circulation.

Apheresis (including plasmapheresis and erythrocytapheresis) can be used for the treatment of conditions in which a pathogenic substance or component in the blood is causing morbidity. In the present invention, the inventors have identified the pathogenic component as RCDP, as described herein.

During the apheresis procedure of the present invention, blood (which consists of blood cells and blood plasma) can initially be taken out of the body through a needle or previously implanted catheter. Plasma can then then removed from the blood by a cell separator. The plasma can be separated by discontinuous flow centrifugation, continuous flow centrifugation, and/or plasma filtration. After plasma separation, the plasma (i.e. which contains the RCDP) can be treated (i.e. to remove the RCDP). After plasma treatment, the plasma can be returned to the patient.

The blood cells (e.g. erythrocytes) can be returned to the person undergoing treatment.

Alternatively, the blood cells can be discarded and replaced with blood cells provided by a blood donor, rather than returning them to the person undergoing treatment. In particular, the erythrocytes can be discarded and replaced with erythrocytes provided by a blood donor, rather than returning them to the person undergoing treatment. The apheresis or plasmapheresis may be red blood cell exchange apheresis or plasmapheresis.

### Subject

The subject described herein may be any suitable subject. The subject may be a human subject.

Suitably, the subject may be a subject with a blood clotting disorder.

The subject may have a sub-optimal plasma clotting time and/or increased hypercoagulability (also known as thrombophilia or a prothrombotic state). Hypercoagulability can be congenital or acquired.

The subject may have a haemoglobinopathy, haemolytic anaemia, and/or an erythrocyte membrane disorder. The subject may have a sickle cell disease.

The subject may be a splenectomised subject.

### Haemoglobinopathy

The subject may have a haemoglobinopathy. In other words, the plasma to be modified in the present invention may be plasma from a subject with a haemoglobinopathy.

Haemoglobinopathy is a hereditary condition involving an abnormality in the structure of haemoglobin. Typically, hemoglobinopathies are inherited single-gene disorders and in most cases, they are inherited as autosomal co-dominant traits.

The highly variable clinical manifestations of the hemoglobinopathies range from mild hypochromic anemia to moderate hematological disease to severe, lifelong, transfusion-dependent anemia with multiorgan involvement (Kohne, E., 2011. Deutsches Ärzteblatt International, 108(31-32), p.532.).

The haemoglobinopathy may be a disease with a structural haemoglobin variant (abnormal haemoglobin) and/or a thalassemia syndrome.

The haemoglobinopathy may be a disease with a structural haemoglobin variant (abnormal haemoglobin). This group of autosomal dominant inherited haemoglobin disorders is caused by structural defects resulting from an altered amino acid sequence in the α or β chains.

Hemoglobinopathies involving structural haemoglobin variants may include one or more of: sickle-cell disease; variants with abnormal haemoglobin synthesis, e.g. HbE; variants with a tendency to precipitate and with haemolysis (unstable haemoglobins), e.g. Hb Köln; and variants with abnormal oxygen transportation and congenital polycythaemia, e.g. Hb Johnstown, or with congenital cyanosis (abnormal methaemoglobin, HbM abnormalities, e.g. M Iwate).

The haemoglobinopathy may be a thalassemia syndrome. Thalassemia syndromes include all thalassemic Hb synthesis disorders. These are autosomal recessive conditions. α- and β-thalassemias have the greatest clinical significance.

The haemoglobinopathy may be α-thalassemia. α-thalassemias are caused by an α-globin chain synthesis defect. The α-thalassemia may be one or more aα-thalassemia selected from: clinically inapparent α-thalassemia (heterozygous α⁺-thalassemia, -α/αα); α-thalassemia minor (heterozygous α⁰-thalassemia, --/αα, or homozygous α⁺-thalassemia, -α/-α); HbH disease (compound heterozygous α⁺/α⁰-thalassemia with three inactive α-genes, --/-α); and Hb Bart's hydrops fetalis (homozygous α⁰-thalassemia).

The haemoglobinopathy may be β-thalassemia. β-thalassemias are the result of insufficient (β⁺) or absent (β⁰) production of β-globin chains. The β-thalassemia may be one or more β-thalassemia selected from: thalassemia minor (heterozygous β-thalassemia); thalassemia intermedia (mild homozygous or mixed heterozygous β-thalassemia); and thalassemia major (severe homozygous or mixed heterozygous β-thalassemia).

### Sickle Cell Disease

Preferably, the subject has a sickle cell disease. In other words, the plasma to be modified in the present invention may be plasma from a subject with a sickle cell disease.

Sickle cell disease (SCD) is a group of inherited disorders caused by mutations in HBB, which encodes haemoglobin subunit β. SCD occurs when both HBB alleles are mutated and at least one of them is the β^{S} allele. (Kato, G. J., et al., 2018. Nature Reviews Disease Primers, 4(1), pp.1-22). SCD is characterised by haemolytic anaemia, hypercoagulability and inflammation. SCD may be diagnosed by any suitable method known by those of skill in the art, for example by genetic screening.

The most common type of SCD is known as sickle cell anaemia (SCA). Individuals with SCA have two βS alleles (β^{S}/β^{S}). Preferably, the sickle cell disease is sickle cell anaemia.

Other relatively common SCD genotypes are possible including HbSβ-thalassemia, or HbSC disease. The sickle cell disease may be HbSβ-thalassemia or HbSC disease.

The β^{S} allele combined with a null HBB allele (Hbβ⁰) that results in no protein translation causes HbSβ⁰-thalassaemia, a clinical syndrome indistinguishable from SCA except for the presence of microcytosis (a condition in which erythrocytes are abnormally small). The β^{S} allele combined with a hypomorphic HBB allele (Hbβ⁺; with a decreased amount of normal β-globin protein) results in HbSβ⁺-thalassaemia, a clinical syndrome generally milder than SCA owing to low-level expression of normal HbA.

Individuals with the HbSC genotype have one β^{S} allele and one HBB allele with a different nucleotide substitution (HBB Glu6Lys, or β^{C} allele) that generates another structural variant of Hb, HbC. HbSC disease is a condition with generally milder haemolytic anaemia and less frequent acute and chronic complications than SCA, although retinopathy and osteonecrosis (also known as bone infarction, in which bone tissue is lost owing to interruption of the blood flow) are common occurrences.

Rarely seen compound heterozygous SCD genotypes include HbS combined with HbD, HbE, HbO^{Arab} or Hb Lepore.

### Haemolytic Anaemia

The subject may have haemolytic anaemia. In other words, the plasma to be modified in the present invention may be plasma from a subject with haemolytic anaemia.

Haemolytic anemia is a form of anemia due to haemolysis, the abnormal breakdown of red blood cells (RBCs), either in the blood vessels (intravascular haemolysis) or elsewhere in the human body (extravascular, but usually in the spleen).

Symptoms of haemolytic anemia can be similar to other forms of anemia (fatigue and shortness of breath), but in addition, the breakdown of red cells can lead to jaundice and can increase the risk of particular long-term complications, such as gallstones and pulmonary hypertension.

The haemolytic anemia may be congenital haemolytic anemia and/or acquired haemolytic anemia.

Congenital haemolytic anemia refers to haemolytic anemia which is hereditary. The congenital haemolytic anemia may be caused by one or more of: defects of red blood cell membrane production (as in hereditary spherocytosis and hereditary elliptocytosis); defects in haemoglobin production (as in thalassemia, sickle-cell disease and congenital dyserythropoietic anemia); defective red cell metabolism (as in glucose-6-phosphate dehydrogenase deficiency and pyruvate kinase deficiency); and paroxysmal nocturnal haemoglobinuria (PNH). The subject may have glucose-6-phosphate dehydrogenase deficiency.

Acquired haemolytic anemia may be caused by immune-mediated causes, drugs and other miscellaneous causes. The acquired haemolytic anemia may be cause by one or more of: immune-mediated haemolytic anemia, for example an autoimmune haemolytic anemia; spur cell haemolytic anemia; any of the causes of hypersplenism (increased activity of the spleen), such as portal hypertension; burns or infection; lead, arsine or stibine poisoning; and footstrike haemolysis.

### Erythrocyte Membrane Disorders

The subject may have an erythrocyte membrane disorder. In other words, the plasma to be modified in the present invention may be plasma from a subject with an erythrocyte membrane disorder.

Disorders of the erythrocyte membrane, including hereditary spherocytosis, hereditary elliptocytosis, hereditary pyropoikilocytosis, and hereditary stomatocytosis, comprise an important group of inherited haemolytic anaemias (Narla, J. and Mohandas, N., 2017. International journal of laboratory hematology, 39, pp.47-52).

The subject may have a disorder which alters the membrane structural organization, such as hereditary spherocytosis, hereditary elliptocytosis, Southeast Asian ovalocytosis. The subject may have a disorder which alters membrane transport function, such as hereditary stomatocytosis and hereditary xerocytosis.

The subject may have spherocytosis. Spherocytosis is a disorder characterised by sphere-shaped rather than bi-concave disk shaped erythrocytes.

The subject may have hereditary spherocytosis. Hereditary spherocytosis is a group of inherited disorders characterised by the presence of spherical-shaped erythrocytes on the peripheral blood smear (Gallagher, P. G., 2005. ASH Education Program Book, 2005(1), pp.13-18).

The subject may have elliptocytosis. Elliptocytosis is a disorder characterised by elliptically-shaped rather than bi-concave disk shaped erythrocytes.

The subject may have hereditary elliptocytosis. Hereditary elliptocytosis is group of inherited disorders characterised by the presence of elliptical, cigar-shaped erythrocytes on peripheral blood smear (Gallagher, P. G., 2005. ASH Education Program Book, 2005(1), pp.13-18).

The subject may have Southeast Asian ovalocytosis. Southeast Asian ovalocytosis is an unusual, dominantly inherited hereditary elliptocytosis variant found in Malaysia, Papua New Guinea, the Philippines, and other parts of Southeast Asia. Rounded elliptocytes, or ovalocytes, and characteristic stomatocytes with longitudinal slits are found on peripheral blood smear (Gallagher, P. G., 2005. ASH Education Program Book, 2005(1), pp.13-18).

The subject may have stomatocytosis. Stomatocytosis is a condition in which a mouth-like or slit-like pattern replaces the normal central zone of pallor in erythrocytes. Stomatocytosis can be congenital (hereditary stomatocytosis) or acquired.

The subject may have hereditary stomatocytosis. Hereditary stomatocytosis is a group of inherited disorders characterized by erythrocytes with a mouth-shaped (stoma) area of central pallor on peripheral blood smear (Gallagher, P. G., 2005. ASH Education Program Book, 2005(1), pp.13-18). The subject may have dehydrated hereditary xerocytosis (dehydrated hereditary stomatocytosis) or overhydrated hereditary stomatocytosis.

The subject may have hereditary pyropoikilocytosis. Hereditary pyropoikilocytosis is an autosomal recessive form of haemolytic anemia characterized by an abnormal sensitivity of red blood cells to heat and erythrocyte morphology similar to that seen in thermal burns or from prolonged exposure of a healthy patient's blood sample to high ambient temperatures.

The subject may have acanthocytosis. Acanthocytosis can refer generally to the presence of acanthocytes, which are a form of red blood cell that has a spiked cell membrane, due to abnormal thorny projections.

### Alloantibodies and Hyperhaemolysis

The subject may have alloantibodies and/or autoantibodies, and/or the subject may have hyperhaemolysis syndrome, especially when the subject has sickle cell disease. In other words, the plasma to be modified in the present invention may be plasma from a subject with alloantibodies and/or autoantibodies, and/or from a subject who has hyperhaemolysis syndrome.

The method of the invention may be of particular benefit to patients for whom transfusion is not possible either because of the presence of multiple alloantibodies or autoantibodies, previous hyperhaemolysis, or because of the lack of availability of a safe blood supply.

The subject may have alloantibodies, suitably multiple alloantibodies, especially when the subject has sickle cell disease. Alloantibodies are antibodies that are produced following exposure to foreign red blood cell antigens. Alloantibodies may be formed in response to pregnancy, transfusion, or transplantation targeted and targeted against a blood group antigen that is not present on the person's red blood cells. Alloantibodies may lead to destruction of transfused red blood cells, meaning a transfusion is not possible.

The subject may have autoantibodies, suitably multiple autoantibodies, especially when the subject has sickle cell disease. Autoantibodies are antibodies that targets antigens present on the patient or donors' own red blood cells. Autoantibodies can result in positive direct antiglobulin tests. Autoantibodies can cause problems in blood bank compatibility testing because they tend to react against a wide array of donor red blood cells, and finding compatible blood may be very difficult.

The subject may have and/or previously have had hyperhaemolysis syndrome, especially when the subject has sickle cell disease (Banks, M. and Shikle, J., 2018. Archives of pathology & laboratory medicine, 142(11), pp.1425-1427). Hyperhaemolysis syndrome is characterised by the development of severe anemia with post transfusion haemoglobin levels that are lower than pre-transfusion levels. As well as sickle cell disease, hyperhaemolysis syndrome has also been reported in patients with other conditions, such as thalassemia. Transfusion may be avoided in patients with hyperhaemolysis syndrome, and even transfusion with genotype-matched red blood cells is not without risk.

### EXAMPLES

### Example 1—SCD Patients Have Elevated Levels of RCDP

Studies utilising flow cytometry have reported elevated numbers of membrane vesicles from different cell sources, in the plasma of patients with SCD (Shet A S, et al. Blood. 2003; 102(7):2678-2683; Westerman M, et al. British journal of haematology. 2008;142(1):126-135). Typically, an antibody recognising an extracellular epitope on GPA is used to distinguish RCDPs from those derived from other cells. Recently autophagic vesicles (AVs) have been shown to be released from maturing reticulocytes using BRIC163, (Okubo Y, et al. Vox Sang. 1988;54(2):107-111) an antibody to the intracellular cytoplasmic domain of GPA (Mankelow T J, et al. Blood. 2015;126(15):1831-1834). In order to detect all RCDP in SCD plasma we dual stained with BRIC256, (Reid M E, et al. Transfusion clinique et biologique. 1997;4(1):57-64) which recognises an extracellular epitope on GPA, and BRIC163, so we could distinguish between right side-out vesicles (BRIC256^{+ve}) and AVs (BRIC163^{+ve}). Imaging flow cytometry permitted visualisation of the different RCDP present and allowed detection of microparticles too small to be detected on a conventional flow cytometer.

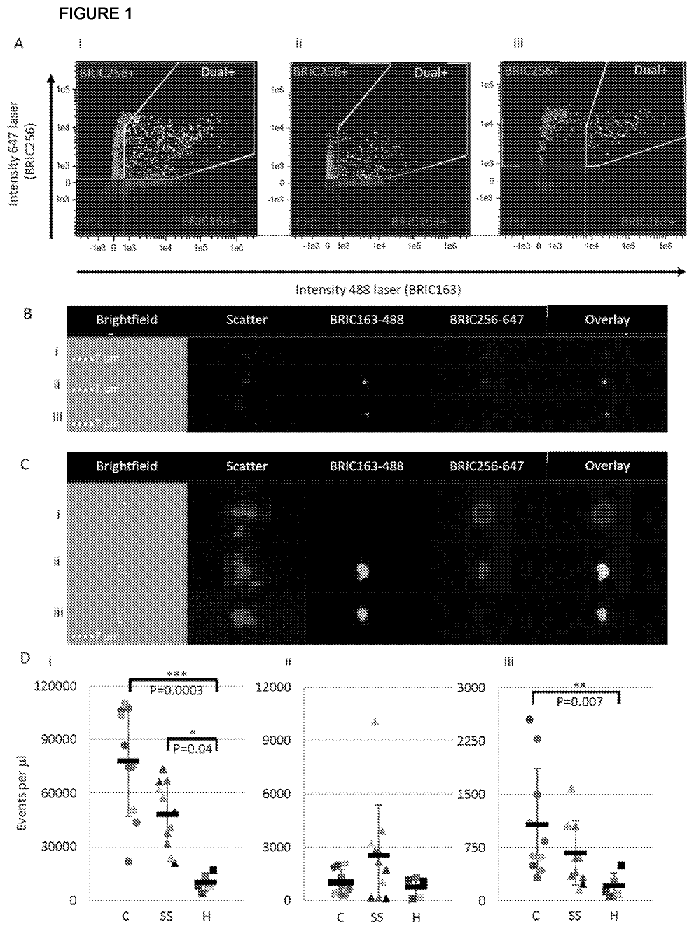

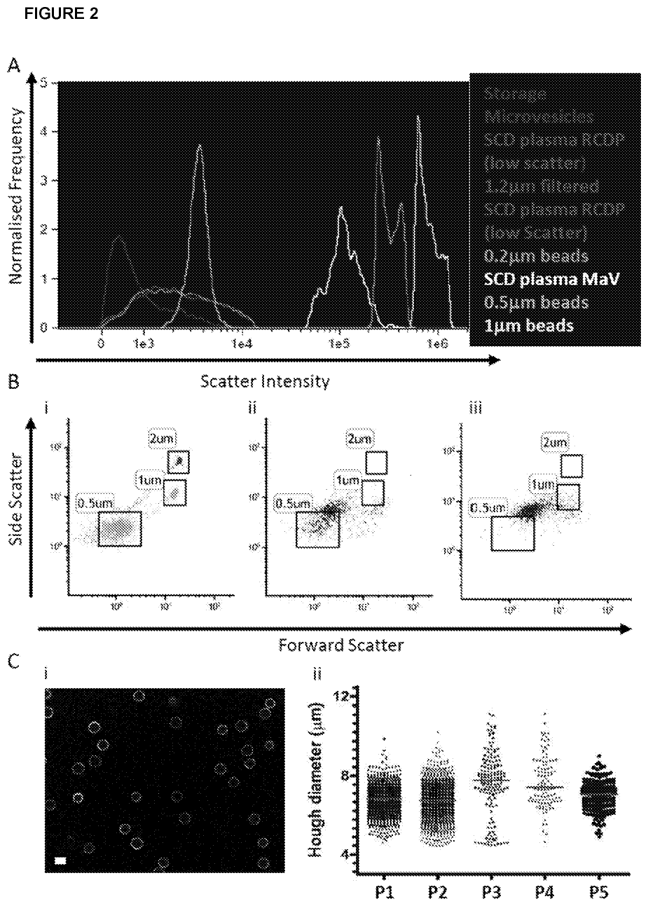

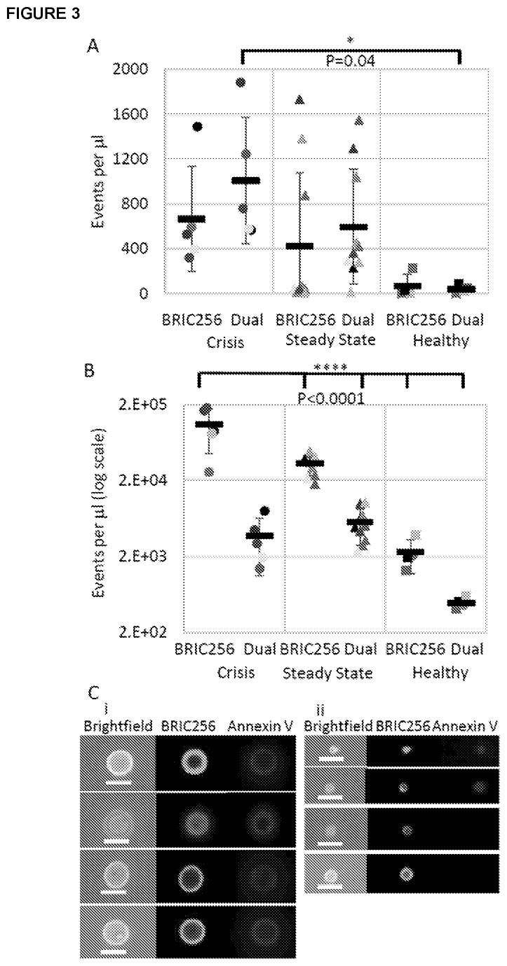

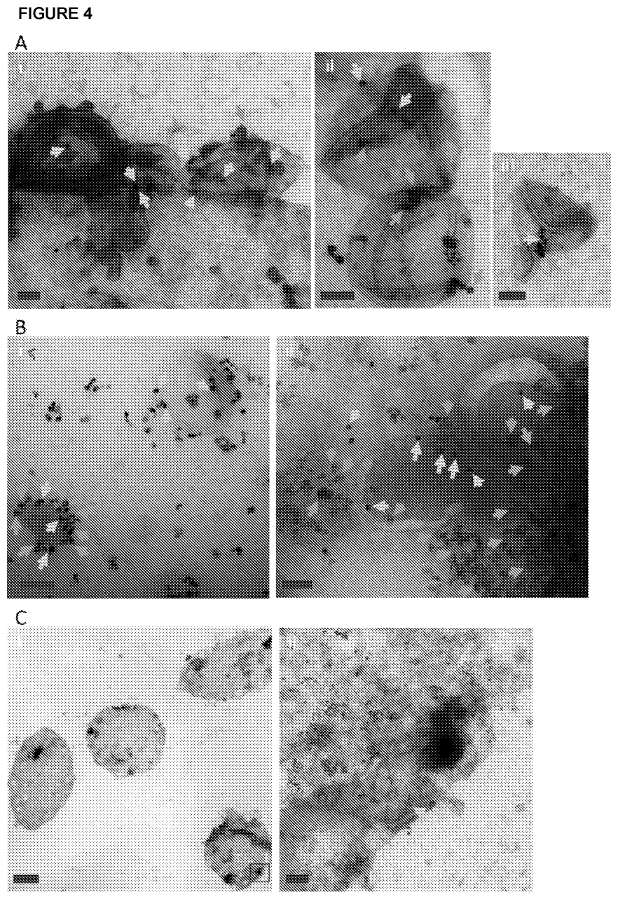

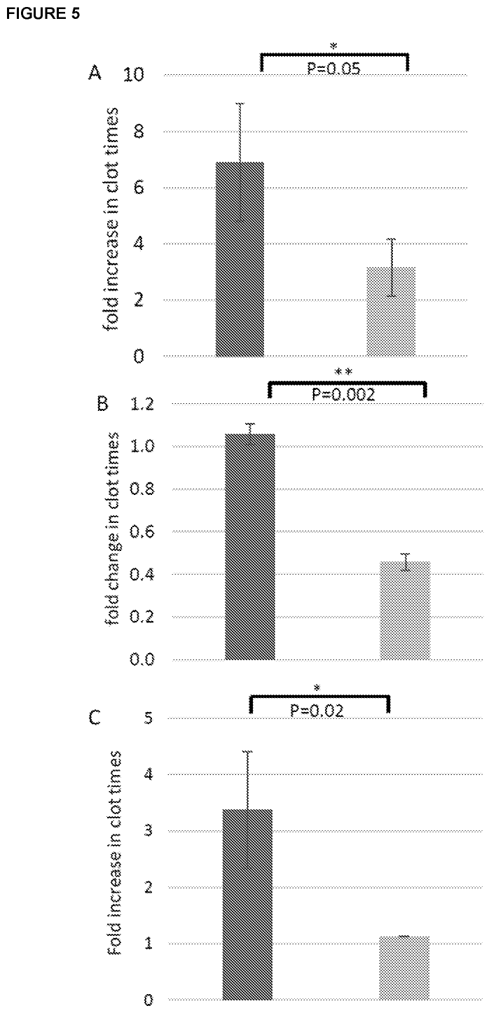

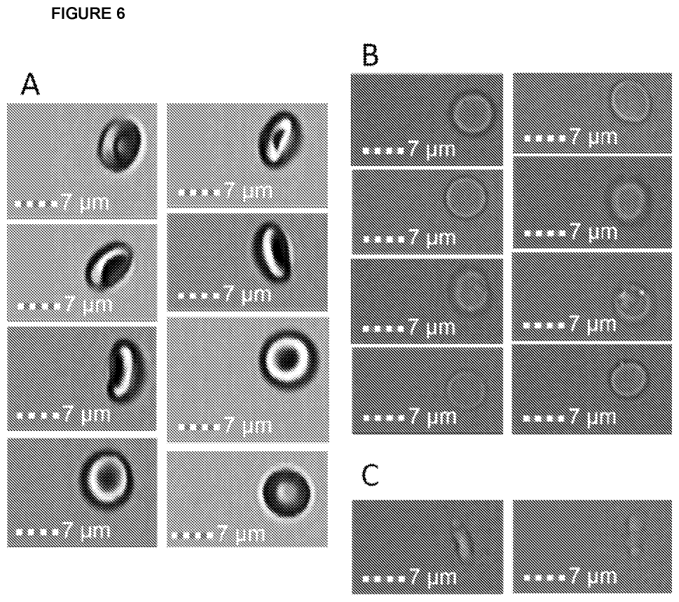

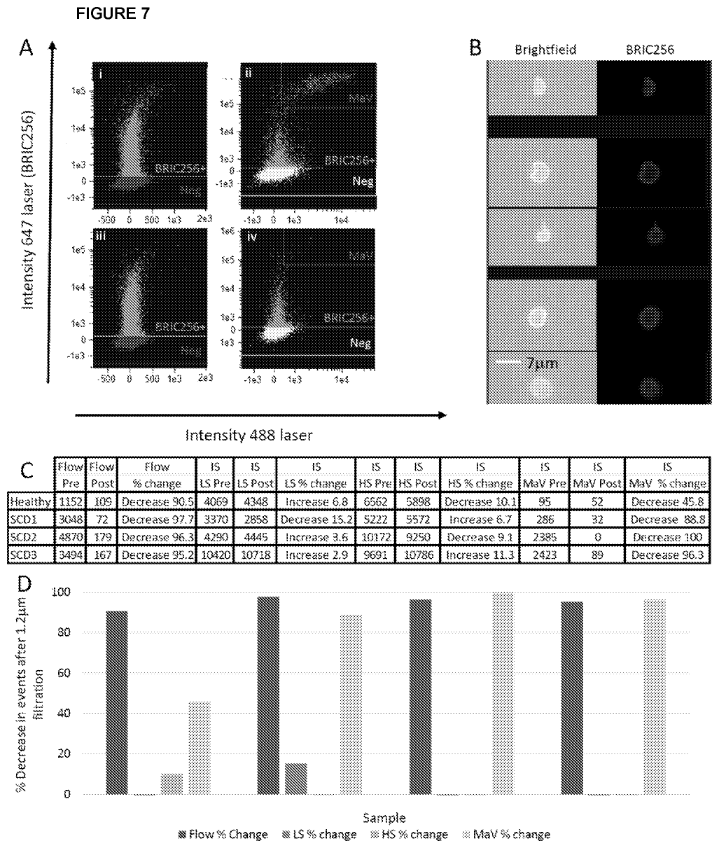

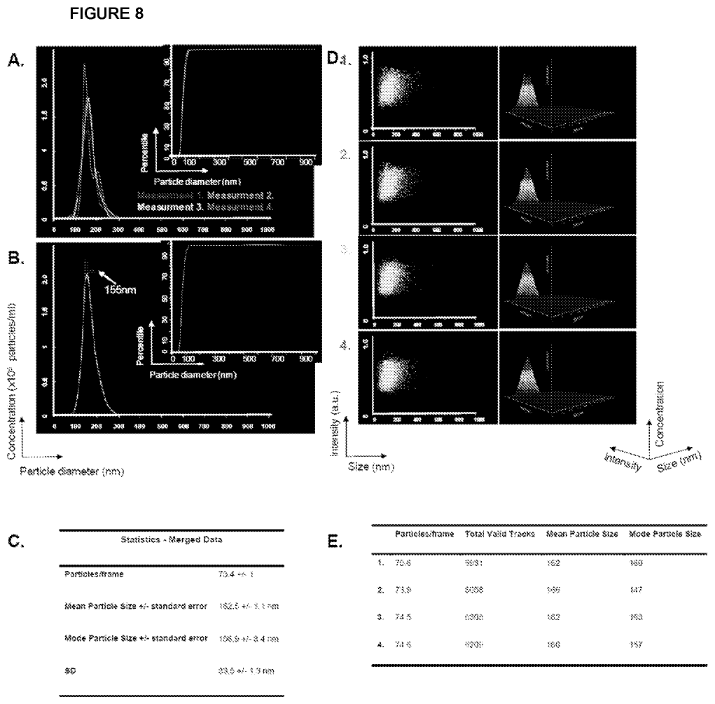

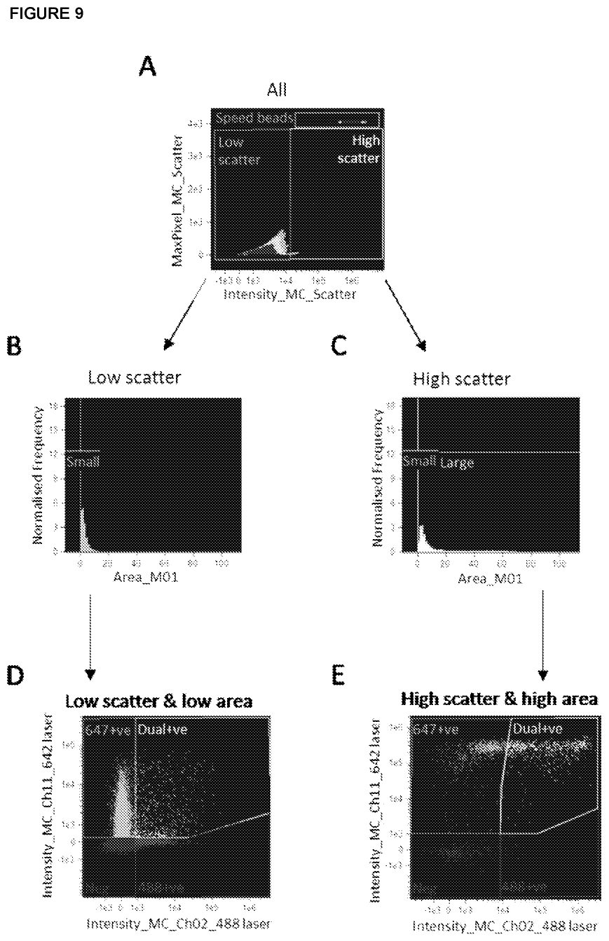

Platelet-free plasma (PFP) from patients with SCD contained a very heterogeneous population of RCDP (FIG. 1). The most abundant population detected corresponded to BRIC256^{+ve} vesicles. Also present were BRIC163^{+ve} AVs and duare events (FIG. 1Ai). The data generated were subdivided into events with low scatter (small vesicles, FIG. 1Aii) and high scatter (larger particles, FIG. 1Aiii). The low scatter particles were predominantly microvesicles (FIG. 1B). The BRIC256^{+ve} low scatter events are likely vesicles released by cellular blebbing during repeated cycles of cellular sickling and recovery. A large proportion of the high scatter BRIC256^{+ve} events (FIG. 1Aiii) correspond to large, erythrocyte sized, particles (approximately 7 μm, FIG. 10). All intact cells were removed from the PFP by centrifugation and the membrane definition, as viewed by brightfield by imaging flow cytometry, of these high scatter events does not correspond to erythrocytes (FIG. 6). It seems likely these particles are erythrocyte macrovesicles (MaV) that have formed in circulation as a result of repeated sickling events. Similar MaV are observed in PFP from healthy individuals but in reduced numbers compared with SCD PFP (a mean of 5 per μl in healthy plasma (n=2) and 826 per μl in steady state SCD plasma (n=4)). Dual BRIC256^{+ve} and BRIC163^{+ve} events are likely to be unsealed membrane fragments from lysed red cells.

We analysed SCD PFP from patients in steady state and in crisis in comparison with PFP from healthy individuals (FIG. 1D). Steady state SCD patients have significantly elevated levels of all RCDP (low and high scatter) with 5-fold more BRIC256^{+ve} RCDP (P=0.04) and 3-fold more BRIC163^{+ve} and dual^{+ve} RCDP than healthy controls. Patients in crisis had significantly higher levels of BRIC256^{+ve} (P≤0.0003) and dual^{+ve} particles than healthy donors (P≤0.007).

Identification and characterisation of RCDP in the plasma of SCD patients have generally employed conventional flow cytometry, however, in this study we used the more sensitive imaging flow cytometry in order to detect and quantify the number and size of RCDP in the plasma of SCD patients. The results show that RCDP are present in a wide range of sizes from MaV and fragments of red cell membrane to microvesicles. AVs are also present and have a much smaller range of sizes and appear to be far less abundant than the other subsets of RCDP. These data demonstrate that circulating in plasma are different types of RCDP rather than a single homogenous population.

We show that there are significant differences in the numbers of RCDP present in the plasma of healthy individuals and SCD patients both in steady state and crisis. SCD patients in crisis have significantly elevated numbers of right side-out RCDP and membrane fragments (BRIC256/BRIC163 dual^{+ve}) in plasma, both formed by the sickling process (FIGS. 1D & 3).

### Example 2—GPA^{+ve} Particles Detected on Standard Flow Cytometers Cannot Be True Microvesicles and Are in Fact Macrovesicles

ImageStream analysis of RCDP in PFP from SCD patients revealed the presence of RCDP diverse in size comprising particles sealed in a right side-out membrane orientation, particles sealed in an inside-out orientation (AV) and unsealed membrane fragments (FIG. 1).

When SCD PFP was analysed by imaging flow cytometry with size beads the scatter intensity of the low scatter population straddles that of 0.2 μm beads and the scatter intensity of the high scatter population is lower, though overlaps with, that of the 0.5 μm beads (FIG. 2A). When SCD PFP was passed through a 1.2 μm filter, a sub-population of the larger high scatter population, the MaV population, was no longer observed in plots of high scatter events (FIGS. 2A and 6). The low scatter particles passed through the filter indicating a size less than 1.2 μm (FIG. 2A, Supple-mental FIG. 2).

A further comparison was undertaken using storage induced RCDP, which were found to be under 200 nm (FIG. 8). When compared with RCDP in PFP, the smaller particles that comprise the low scatter events overlap with the storage induced RCDP but appear to have a slightly higher scatter (FIG. 2A). This is also consistent with the size of the low scatter events when viewed under brightfield (FIG. 1C). The larger high scatter particles have more scatter than the storage induced RCDP but a lower scatter than 0.5 μm beads (FIG. 2A), even though they are clearly much larger when viewed on brightfield (FIG. 1Ci).

This suggests that previous studies analysing RCDV present in SCD PFP by standard flow cytometry (Shet A S, et al. Blood. 2003;102(7):2678-2683; Simak J, et al. British journal of haematology. 2004;125(6):804-813; and Westerman M, et al. British journal of haematology. 2008;142(1):126-135) using size beads and/or scatter to determine gates, were likely detecting these large MaV, rather than the whole population of RCDP present. Indeed, when we analysed SCD PFP by standard flow cytometry with size beads (Beckman Coulter Navios) we were unable to detect either the ImageStream low scatter events or storage induced RCDP and predominantly detected the high scatter MaV population observed on the ImageStream (FIG. 2B, FIG. 7). In addition, 1.2 μm filtration of SCD PFP resulted in an almost complete loss of RCDP detected by standard flow cytometry. However, with Imaging flow cytometry it is only the MaV population that is lost with 1.2 μm filtration while the low scatter and a small subset of the high scatter population remains (FIG. 2A and FIG. 7). This result is further evidence that size beads are an inaccurate method for determining the size of vesicles by flow cytometry (Lannigan J, Erdbruegger U. Methods. 2017;112:55-67).

Further characterisation of MaV was undertaken by spinning-disk confocal analysis. This technique is not sensitive enough to detect the low scatter RCDP population and the smaller sized particles of the high scatter population of RCDP, however, it can detect the MaV population. Images showed that FACS sorted GPA^{+ve} MaV were mainly intact and circular, rather than blebs or broken membrane parts (FIG. 2Ci). In order to calculate the size and radial symmetry of these particles, images from five SCD patients were analysed using the Hough circle transform and found to have a median diameter of 7 μm (range 6.8-7.8 μm (FIG. 2C, Table 1). Therefore, MaV are too large to be considered microvesicles, since they have a similar size to erythrocytes. These findings are also in accordance with the imaging flow cytometry results (FIGS. 1, 2, 6 and 7), suggesting that GPA^{+ve} particles detected on standard flow cytometers cannot be true microvesicles and are in fact macrovesicles (MaV).

Comparison of the results from imaging flow cytometry with those from conventional flow cytometry leads us to conclude that RCDP detected by many conventional flow cytometers are in fact not microvesicles but larger macrovesicles (MaV) (FIGS. 1, 6 and 7). This misconception can be attributed to anomalous behaviour of beads, possibly due to differences in the light scattering intensity between lipid particles and polystyrene beads.

TABLE 1
Spinning-disk confocal analysis of BRIC256
FACS sorted steady state SCD samples.
Scatter plot of size of each individual round object
detected in each sample are shown in FIG. 2C. Data
range, median and quartiles are shown in Table 1.

| | P1 | P2 | P3 | P4 | P5 |
|---|---|---|---|---|---|
| Number of Values | 1332 | 1547 | 150 | 113 | 183 |
| Minimum | 4.6 | 4.4 | 4.4 | 4.6 | 4.9 |
| 25% Percentile | 6.4 | 6.2 | 6.8 | 6.7 | 6.5 |
| Median | 6.8 | 6.7 | 7.8 | 7.4 | 7.0 |
| 75% Percentile | 7.2 | 7.2 | 8.5 | 8.6 | 7.5 |
| Maximum | 9.9 | 10.2 | 11.1 | 11.2 | 9.0 |

### Example 3—Characterisation of RCDP

Phosphatidylserine (PS) is usually found on the inner leaflet of the plasma membrane. It is a known pro-thrombotic and pro-inflammatory signal and an “eat me” signal for professional phagocytic cells (Ravichandran K S. Immunity. 2011;35(4):445-455). As such, any unregulated exposure can lead to physiological problems and elevated levels of exposed PS have been associated with the pathologies of a number of diseases and in particular SCD (Wood B L, et al. Blood. 1996;88(5):1873-1880; Kuypers F A, et al. Blood. 1998;91(8):3044-3051; and Kuypers F A, et al. Blood. 1996;87(3):1179-1187).

To establish whether the different populations of RCDP observed by imaging flow cytometry were PS^{+ve}, PFP from SCD patients, in steady and state and crisis, along with healthy controls were dual stained with BRIC256 and Annexin V (FIG. 3). The majority of the BRIC256 high scatter events and all the MaV sub-population are PS^{+ve} (dual^{+ve}, FIG. 3A and C), as detected by Annexin V. Similar PS decorated large membrane structures have previously been observed by electron microscopy in low numbers in healthy plasma (Arraud N, et al. J Thromb Haemost. 2014;12(5):614-627), however, here we show their numbers are significantly elevated in the plasma of SCD patients in crisis compared to healthy subjects (P=0.04).

The large size (7 μm) and number (average 1000/μl in crisis patients (n=5), average 590/μl steady state (n=10)) of these PS decorated MaV would make them likely candidates for involvement in the pathology of SCD. Low scatter BRIC256^{+ve} particles were more numerous, particularly in SCD patients in crisis (P<0.0001, FIG. 3B). However, significantly fewer particles were also PS^{+ve}, regardless of the plasma source (P≤0.04). The failure of the low scatter small particles to bind Annexin V may be due to the high curvature of these sub-cellular particles rather than an absence of PS (Shi J, Gilbert G E. Blood. 2003;101(7):2628-2636).

After magnetic isolation of the right-side and inside-out vesicles, the morphology of the populations was examined by TEM. In both types of vesicles, round structures were observed with an external lipid bilayer (FIG. 4). Magnetic beads (˜50 nm) appeared as roundish, grey structures and gold particles (˜10 nm) as black dots. Enriched right side-out vesicles were stained with BRIC256 or BRIC200. Isolated inside-out microvesicles were already stained, as part of the isolation protocol. As previously observed with imaging flow cytometry (FIGS. 1 and 2), there is a great diversity in size amongst RCDP found in the plasma of SCD patients. The TEM results show that the right side-out and inside-out (stained only with BRIC256 or BRIC163, not both) RCDP are structures with an intact plasma membrane.

Our data indicate that hypercoagulation in SCD patients may primarily result from large side out MaV with exposed PS, generated by repeated sickling events. Compared with the other RCDP detected, this population of MaV is present in fewer numbers in SCD plasma, although still in significantly greater numbers than in healthy individuals (FIG. 3A). However, with a cellular size of approximately 7 μm in diameter, they are by far the largest. Therefore, MaV result in a considerable amount of PS exposed membrane being present in the plasma of SCD patients. In addition, PS exposed membranes have been demonstrated to act as surfaces for complement deposition and thus activation of the immune system. As a result, these large PS exposed particles as well as being pro-thrombotic, may also be pro-inflammatory and as such they may contribute greatly to the pathology of SCD.

### Example 4—Removal of RCDP Increased Clotting Times

We investigated whether the BRIC256^{+ve} particles influenced PFP clotting times. Magnetic beads were used to clear SCD PFP of all right side-out vesicles, in addition, the impact of adding these isolated vesicles to PFP from healthy individuals was also determined. Clotting times before and after each stage were assessed (FIG. 5). Removing right side-out RCDP from SCD PFP increased the clotting times 7-fold whereas removing them from healthy PFP increased clotting times by only 3-fold (FIG. 5A). Moreover, placing right side-out RCDP from SCD PFP into PFP from healthy individuals decreased the clotting time by half, whereas the addition of the magnetic beads only had no effect (FIG. 5B).

As it is the MaV that predominately expresses PS it is likely that this sub-population of RCDP is the most prothrombotic in SCD plasma. Therefore, we assayed clotting times after filtration through a 1.2 μm filter to remove the MaV population (FIGS. 1 and 6) but retain the other RCDP populations. Removal of the MaV population increased clotting times (FIG. 5C) by approximately half the amount that removal of the entire RCDP population (FIG. 5A), while there was no effect on the clotting time of healthy samples.

This suggests that PS decorated RCDP in plasma are prothrombotic and that in SCD, where there are large numbers of these RCDP, they will significantly contribute to the hypercoagulative state.

Removal of released, acellular AV using antibodies to cytoplasmic domains of red cell membrane proteins did not have a significant effect on clotting time. This may be because of the much lower abundance of AV derived particles in SCD plasma compared to the other populations of red cell derived particles.

We isolated RCDP from the plasma of SCD patients by immunoaffinity absorption and demonstrated a dramatic increase in clotting times. RCDP from SCD plasma also provoked a considerable shortening of clotting time when added to normal healthy plasma, demonstrating a direct link between right side-out red cell vesicles and hypercoagulation in SCD patients.

We conclude that RCDP could offer a measure of disease activity and their removal from the plasma in people with SCD could reduce the frequency and severity of vaso-occlusive episodes and provide a much-needed additional therapeutic intervention, improving the quality of life of patients with SCD. Such a procedure may be of particular benefit to patients for whom transfusion is not possible either because of the presence of multiple alloantibodies or previous hyperhemolysis or because of the lack of availability of a safe blood supply, the latter being the case for most patients with this condition internationally.

### Example 5—Methods

### Patients

SCD Patients presenting in a ‘steady state’ or ‘in crisis’ were eligible for inclusion in the study. Steady state was defined as “attending the clinic for a routine red cell exchange transfusion and not having had a transfusion in the previous 4 weeks”. In crisis was defined as “presenting to the Emergency Department in pain”. Blood samples were drawn immediately prior to any transfusion procedure and anonymised. All patients gave informed written consent and the study was approved. Plasma used for electron microscopy and spinning disk confocal analyses came from surplus blood from anonymised samples taken from patients in steady state for clinical haematological analysis from SCD patients.

### Samples

Blood samples were drawn into vacutainers containing sodium citrate. Within 1 hour, plasma and cells were separated after centrifugation (2000 g for 15 minutes at room temperature with no brake) and the plasma stored at 4° C. for under 48 hours. After transportation the plasma was further centrifuged twice (2500g for 20 minutes at room temperature) and the pellet discarded after each step. Approximately 100 μl supernatant was left in the tube after each centrifugation to avoid disturbing the pellet. The platelet free plasma (PFP) was aliquoted and stored at −80° C. until analysis when it was thawed at 37° C. and used immediately. Control samples were drawn from healthy volunteers and processed identically.

### Monoclonal Antibodies

BRIC256 (Reid M E, et al. Transfusion clinique et biologique 1997;4(1):57-64) and R10 (Anstee D J, Edwards P A. Eur J Immunol. 1982;12(3):228-232) have an extracellular glycophorin A (GPA) epitope. BRIC163 (van Tits L J, et al. Biochem Biophys Res Commun. 2009;390(1):161-164) has an intracellular GPA epitope. BRAC66 (Beckmann R, et al. Mol Membr Biol. 2002;19(3):187-200), BRIC155 and BRIC132 (Wainwright S D, et al. Biochem J. 1989;258(1):211-220) have intracellular AE-1 epitopes.

### ImageStream Analysis

Antibodies (BRIC163 and BRIC256) were labelled with either Alexa Fluor-488 or Alexa Fluor-647. 20 μl of platelet free plasma (PFP) was mixed with either an equal volume of 0.22 μm filtered flow-buffer (PBS 1% -, BSA, 0.05% sodium azide (all Sigma-Aldrich, UK)), containing fluorescent labelled antibodies or 80 μl of 0.22 μm filtered Annexin V binding buffer (Annexin-V-FLUOS Staining Kit, Sigma-Aldrich) containing fluorescent Annexin V. Samples were left for one hour on ice before analysis on an ImageStream®X Mark II imaging flow cytometer (Luminex, USA) using the INSPIRE acquisition software ISX. Samples were run with a low flow rate but high sensitivity, 60× magnification and a minimum of 100,000 events (up to 500,000) acquired. Speed beads were excluded from acquisition and all lasers were set to full power. Unstained samples were used as negative controls. Buffer only and buffer plus antibody controls were run to test background signal. Sizing was performed using a flow cytometry sub-micron reference kit (Thermo Fisher Scientific, UK). Data analysis was performed using IDEAS 6.2 software and fluorescent gates were set using fluorescence minus one controls (see supplemental methods).

### ImageStream®X Mark II Imaging Flow Cytometry Data Analysis Using IDEAS 6.2

ImageStream data was analysed using IDEAS version 6.2 (Luminex). Raw image files were opened and a scatter plot of scatter intensity vs scatter max pixel was created to exclude speed beads from analysis and define low scatter and high scatter populations (FIG. 9A). To clean up these populations, ensuring that no large aggregates were present in the “low scatter” population and no small debris was present in the “high scatter” population, histograms of brightfield area were set up to exclude large area events and small area events from the “low scatter” and “high scatter” populations respectively (Supplemental FIGS. 9B and 9C).

Scatter plots were then created using the “low scatter-low area” gate, representing low scatter events, and the “high scatter-high area” gate, representing high scatter events.

Scatter plots of channel 02 vs channel 11 fluorescence intensity were set up (Channel02 for Alexa Fluor 488 and channel11 for Alexa Fluor 647). Gates were placed on these scatter plots using fluorescence minus one controls. Gated populations were observed in the image gallery to visually confirm that the gates were placed optimally.

Statistics were used from the two final scatter plots to obtain the number of “high scatter” and “low scatter” events that were positive for the respective antibodies or Annexin V (conjugated to Alexa Fluor-488, Alexa Fluor-647). To calculate the number of events per μl, the volume of the sample processed by the ImageStream was obtained from Tools>Sample information>Focus/Fluidics.

### Flow Cytometric Analysis and Cell Sorting

Samples were stained as described above for ImageStream analyses. Flow cytometric analyses were performed using a Navios flow cytometer (Beckman Coulter, UK) with Kaluza Version 1.2. GPA^{+ve} cells were sorted using an Influx high speed fluorescence activated cell sorter with BD Sortware 1.2.0 (BD Biosciences, UK). Sort gates were based on fluorescence minus one controls.

### Isolation of RCDP

RCDP were isolated by magnetic bead separation. Right side-out RCDP were removed from SCD PFP using anti-CD235a (Glycophorin A (GPA)) MicroBeads (Miltenyi Biotec, UK). Inside-out RCDP were removed using anti-Cy5/Alexa Fluor-647 MicroBeads (Miltenyi Biotec) in conjunction with BRIC155, BRIC163 or BRIC132, conjugated to Alexa Fluor-647. Antibodies were filtered through a 15 nm filter (Whatman International Ltd, UK) to remove aggregates. Microbeads (40 μl) and, if appropriate, 5 μg BRIC155, BRIC163 or BRIC132 were added to 800 μl of SCD PFP and incubated with rotation at 4° C. for 30 minutes. PFP and beads were passed through MS columns (Miltenyi Biotec), in a magnetic field, under gravity and collected. For clotting assays, RCDP retained on the column were eluted by flushing 800 μl of PFP from a healthy individual through the column outside the magnetic field. For electron microscopy, RCDP were removed using 300 μl of PBS buffer containing 0.5% (v/v) HSA (Irvine Scientific, Ireland) and 0.6% (v/v) CPD (Sigma-Aldrich). Large RCDP were removed by syringe driven filtration of PFP through a 1.2 μm polyethersulfone acrodisc filter (PALL Life Sciences, UK).

### Clotting Assay

200 μl of PFP was placed in a clear tube at 37° C. and left for 5 minutes. The time taken for a visible clot to form was timed. Clotting was activated by the addition of 20 mM Ca²⁺ and 1 μg of Factor Xa (New England Biolabs, UK) and PFP was swirled gently until a clot had visibly formed. Clotting times given for each sample was the average of three assays.

### Spinning-Disk Confocal Microscopy

Microscopy plates (384 MatriCal MGB101-1-2-LG- BiomatriCal) were coated in poly-L-lysine (Sigma-Aldrich) prior to addition of GPA^{+ve} RCDP, then examined using an Opera LX HCS spinning-disk confocal microscope (Perkin Elmer, UK) with a 60× (NA 1.2) water-immersion lens and analysed using Acapella software. Digital images were visualised using ImageJ. The size and shape of RCDP were determined using a custom MATLAB script. The script uses a built-in circular Hough transform in XY axes to detect particles. The 3D shape was determined by fitting an active contour to the radial average of the image, centred on the central Z-axis of each particle. The Hough transform tool is selective for objects with a high degree of radial symmetry and will ignore ellipses.

### Transmission Electron Microscopy and Immunolabelling (TEM)

Three microlitres of enriched right-side or inside-out RCDP were placed on a carbon coated copper grid, left to dry, then fixed in 4% (v/v) paraformaldehyde (Sigma-Aldrich) with 0.05% (v/v) glutaraldehyde (Agar Scientific, UK) at room temperature for 30 minutes. RCDP were washed in PBS, quenched in 20 mM glycine (Thermo Fisher Scientific) for 10 minutes and incubated with 1% acetylated BSA (Aurion, The Netherlands) in PBS for 10 minutes to block non-specific binding. Grids were placed in R10 (for right side-out vesicles) or BRAC66 (for inside-out vesicles), for 1 hour at room temperature then washed in 0.1% BSA/PBS. Aurion conventional gold reagents (particle size 10 nm) were added to the grids. Anti-mouse gold particles were used for right side-out RCDP and anti-rat gold particles were used for inside-out vesicles. Unbound gold was removed by washing. Grids were counterstained with a solution of 0.3% (w/v) uranyl acetate (Biolab, UK) in 1.8% methylcellulose (Sigma-Aldrich) for 10 minutes on ice then air-dried using the wire loop method. Grids were examined on a Tecnai12 120 kV BioTwin Spirit transmission electron microscope (FEI Company, The Netherlands) and visualised using a FEI CETA camera and TIA software.

All publications mentioned in the above specification are herein incorporated by reference. The publications discussed herein are provided solely for their disclosure prior to the filing date of the present application. Nothing herein is to be construed as an admission that such publications constitute prior art to the claims appended hereto.

Various modifications and variations of the disclosed methods, cells, compositions and uses of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

Various preferred features and embodiments of the present invention will now be described with reference to the following numbered paragraphs (paras).

1. A method of decreasing hypercoagulability and/or increasing plasma clotting time comprising removing red cell-derived particles (RCDP) from plasma isolated from a subject to provide modified plasma.

2. A method of decreasing hypercoagulability, increasing plasma clotting time, and/or treating a haemoglobinopathy, haemolytic anaemia or an erythrocyte membrane disorder in a subject, wherein the method comprises:
(i) isolating plasma from the subject;
(ii) removing RCDP from the plasma to provide modified plasma; and
(iii) administering the modified plasma to the subject.

3. A method according to para 2, wherein one or more of steps (i)-(iii) are performed as part of an apheresis procedure, such as a plasmapheresis procedure.

4. A method according to any one of paras 1 to 3, wherein the RCDP comprise or consist of erythrocyte or reticulocyte-derived micelles, vesicles and/or membrane fragments.

5. A method according to any one of paras 1 to 4, wherein the RCDP have on their surface one or more markers from the membrane of erythrocytes or reticulocytes, preferably wherein the one or more markers are from the extracellular membrane surface of erythrocytes or reticulocytes.

6. A method according to any one of paras 1 to 5, wherein the RCDP have on their surface one or more types of blood group active protein.

7. A method according to any one of paras 1 to 6, wherein the RCDP have on their surface one or more types of blood group active protein selected from the list consisting of: Glycophorin A (GPA), anion exchanger 1 (AE1), glucose transporter 1 (GLUT1), and Glycophorin C (GPC), preferably wherein the RCDP have on their surface GPA.

8. A method according to any one of paras 1 to 7, wherein the RCDP have on their surface the extracellular domains of one or more types of blood group active protein.

9. A method according to any one of paras 1 to 8, wherein the RCDP have on their surface one or more markers selected from the list consisting of: the extracellular domain of GPA, the extracellular domain AE1, the extracellular domain GLUT1, and the extracellular domain GPC, preferably wherein the RCDP express on their surface the extracellular domain of GPA.

10. A method according to any one of paras 1 to 9, wherein the RCDP have a particle diameter of from 0.1 μm to 12 μm, preferably 1.2 μm to 12 μm, more preferably 3 μm to 12 μm.

11. A method according to any one of paras 1 to 10, wherein the RCDP have a median particle diameter of 3-12 μm, preferably 6-9 μm or 6-8 μm, more preferably about 7 μm.

12. A method according to any one of paras 1 to 11, wherein the RCDP express on their surface the extracellular and/or cytoplasmic domain of GPA and have a median diameter of 6-9 μm, 6-8 μm, or about 7 μm.

13. A method of decreasing hypercoagulability and/or increasing plasma clotting time comprising removing vesicles which express on their surface the extracellular and/or cytoplasmic domain of GPA and have a median diameter of 6-9 μm, 6-8 μm, or about 7 μm, from plasma isolated from a subject, to provide modified plasma.

14. A method according to any one of paras 1 to 13, wherein the RCDP have on their surface phosphatidylserine (PS).

15. A method according to any one of paras 1 to 14, wherein the RCDP are removed by affinity chromatography and/or size exclusion chromatography.

16. A method according to any one of paras 1 to 15, wherein the RCDP are removed by a molecule which selectively binds one or more markers expressed on the surface of the RCDP.

17. A method according to para 16, wherein the one or more markers are from the extracellular and/or intracellular membrane surface of erythrocytes or reticulocytes, preferably wherein the one or more markers are from the extracellular membrane surface of erythrocytes or reticulocytes.

18. A method according to para 16 or para 17, wherein the one or more markers are selected from the list consisting of: GPA, AE1, GLUT1, GPC, and PS, preferably wherein the marker is the extracellular and/or cytoplasmic domain of GPA.

19. A method according to any one of paras 16 to 18, wherein the molecule is an antibody or an antibody fragment, preferably an antibody.

20. A method according to any one of paras 16 to 19, wherein the molecule is immobilised.

21. A method according to any one of paras 16 to 20, wherein the molecule is immobilised on the stationary phase of an affinity column.

22. A method according to any one of paras 16 to 21, wherein the molecule is immobilised on a bead, preferably wherein the molecule is immobilised on a magnetic bead.

23. A method according to any one of paras 1 to 15, wherein the RCDP are removed by filtration.

24. A method according to para 23, wherein the filter has a filter size of 0.1-2 μm, 0.5-2 μm, 0.8-1.6 μm, or 1.0-1.5 μm, preferably about 1.2 μm.

25. A method according to any one of paras 1 to 24, wherein 10% or more, 20% or more, 30% or more, 40% or more, 50% or more, 60% or more, 70% or more, 80% or more, 90% or more, or about 100% of the RCDP are removed, preferably about 90% or more of the RCDP are removed.

26. A method according to any one of paras 1 to 25, wherein the modified plasma has 90% or less, 80% or less, 70% or less, 60% or less, 50% or less, 40% or less, 30% or less, 20% or less, 10% or less RCDP compared to the plasma, preferably 10% or less RCDP compared to the plasma.

27. A method according to any one of paras 1 to 26, wherein the modified plasma is substantially free of RCDP.

28. A method according to any one of paras 1 to 27, wherein the modified plasma has a clotting time at least two times longer than the plasma, preferably at least three times longer than the plasma, most preferably at least five times longer than the plasma.

29. A method according to any one of paras 1 to 28, wherein the modified plasma has a clotting time approximately the same as the clotting time for control plasma from a healthy subject.

30. A method according to any one of paras 1 to 29, wherein the plasma is isolated from a subject with a haemoglobinopathy, haemolytic anaemia and/or an erythrocyte membrane disorder.

31. A method according to any one of paras 2 to 30, wherein the haemoglobinopathy is sickle cell disease, preferably wherein the sickle cell disease is sickle cell anaemia, HbSβ-thalassemia, or HbSC disease, more preferably wherein the sickle cell disease is sickle cell anaemia.

32. A method according to any one of paras 2 to 31, wherein the erythrocyte membrane disorder is selected from one or more of hereditary spherocytosis, hereditary stomatocytosis, hereditary elliptocytosis, hereditary pyropoikilocytosis, Southeast Asian ovalocytosis, acanthocytosis, and stomatocytosis.

33. A method according to any one of paras 2 to 32, wherein the haemolytic anaemia is caused by glucose-6-phosphate dehydrogenase deficiency.

34. A method according to any one of paras 1 to 33, wherein the plasma is platelet-free plasma and/or the plasma is fresh plasma or fresh frozen plasma.

35. Modified plasma obtained or obtainable by a method according to any one of paras 1 to 34.

36. Plasma which is substantially free of RCDP which express the extracellular domain of GPA on their surface and have a median diameter of 6-9 μm or about 7 μm.

37. Plasma according to para 36, wherein the plasma is fresh plasma or fresh frozen plasma.

38. An affinity column comprising a stationary phase which specifically binds one or more markers expressed on the surface of RCDP.

39. An affinity column according to para 38 wherein the one or more markers are from the extracellular and/or intracellular membrane surface of erythrocytes or reticulocytes, preferably the one or more markers are from the extracellular membrane surface of erythrocytes or reticulocytes.

40. An affinity column according to para 38 or para 39 wherein the one or more markers are selected from the list consisting of: GPA, AE1, GLUT1, GPC, and PS.

41. An affinity column according to any one of paras 38 to 40 wherein the marker is the extracellular and/or cytoplasmic domain of GPA.

42. An affinity column according to any one of paras 38 to 41 wherein the molecule is an antibody or an antibody fragment, preferably an antibody.

43. An apheresis device or an apheresis kit comprising a molecule or a bead which specifically binds one or more antigens markers expressed on the surface of RCDP, and/or an affinity column according to any one of paras 38 to 42.

44. Use of a molecule or a bead which specifically binds one or more antigens markers expressed on the surface of RCDP, an affinity column according to any one of paras 38 to 42, an apheresis device or an apheresis kit according to para 43, for removing RCDP, for decreasing hypercoagulability, and/or for increasing plasma clotting time.

45. Use of a molecule or a bead which specifically binds one or more antigens markers expressed on the surface of RCDP, an affinity column according to any one of paras 38 to 42, or an apheresis device or an apheresis kit according to para 43, in a method according to any one of paras 1 to 35.

46. A method according to any one of paras 1 to 34, or use according to para 44 or para 45, wherein the plasma clotting time is the Factor Xa-activated clotting time, the thrombin time, the activated partial thromboplastin time, the prothrombin time, or the activated clotting time, preferably the plasma clotting time is the Factor Xa-activated plasma clotting time.

47. A method according to any one of paras 1 to 34 or para 46, or use according to any one of paras 44 to 46, wherein the plasma clotting time is increased by at least two-fold, at least three-fold, or at least five-fold.

1. A method of decreasing hypercoagulability and/or increasing plasma clotting time comprising removing red cell-derived particles (RCDP) from plasma isolated from a subject to provide modified plasma.

2. A method of decreasing hypercoagulability, increasing plasma clotting time, and/or treating a haemoglobinopathy, haemolytic anaemia or an erythrocyte membrane disorder in a subject, wherein the method comprises:
(i) isolating plasma from the subject;
(ii) removing RCDP from the plasma to provide modified plasma; and
(iii) administering the modified plasma to the subject.

3. A method according to any one of claims 1 to 2, wherein the RCDP comprise or consist of erythrocyte or reticulocyte-derived micelles, vesicles and/or membrane fragments.

4. A method according to any one of claims 1 to 3, wherein the RCDP have on their surface one or more markers from the membrane of erythrocytes or reticulocytes, preferably wherein the one or more markers are from the extracellular membrane surface of erythrocytes or reticulocytes.

5. A method according to any one of claims 1 to 4, wherein the RCDP have on their surface one or more types of blood group active protein.

6. A method according to any one of claims 1 to 5, wherein the RCDP have on their surface one or more types of blood group active protein selected from the list consisting of: Glycophorin A (GPA), anion exchanger 1 (AE1), glucose transporter 1 (GLUT1), and Glycophorin C (GPC), preferably wherein the RCDP have on their surface GPA.

7. A method according to any one of claims 1 to 6, wherein the RCDP have a particle diameter of from 0.1 μm to 12 μm, preferably 1.2 μm to 12 μm, more preferably 3 μm to 12 μm.

8. A method according to any one of claims 1 to 7, wherein the RCDP have a median particle diameter of 3-12 μm, preferably 6-9 μm or 6-8 μm, more preferably about 7 μm.

9. A method of decreasing hypercoagulability and/or increasing plasma clotting time comprising removing vesicles which express on their surface the extracellular and/or cytoplasmic domain of GPA and have a median diameter of 6-9 μm, 6-8 μm, or about 7 μm, from plasma isolated from a subject, to provide modified plasma.

10. A method according to any one of claims 1 to 9, wherein the RCDP have on their surface phosphatidylserine (PS).

11. A method according to any one of claims 1 to 10, wherein the RCDP are removed by affinity chromatography and/or size exclusion chromatography.

12. A method according to any one of claims 1 to 11, wherein the RCDP are removed by a molecule which selectively binds one or more markers expressed on the surface of the RCDP.

13. A method according to claim 12, wherein the molecule is an antibody or an antibody fragment, preferably an antibody.

14. A method according to claim 12 or claim 13, wherein the molecule is immobilised.

15. A method according to any one of claims 1 to 11, wherein the RCDP are removed by filtration.

16. A method according to claim 15, wherein the filter has a filter size of 0.1-2 μm, 0.5-2 μm, 0.8-1.6 μm, or 1.0-1.5 μm, preferably about 1.2 μm.

17. A method according to any one of claims 1 to 16, wherein 10% or more, 20% or more, 30% or more, 40% or more, 50% or more, 60% or more, 70% or more, 80% or more, 90% or more, or about 100% of the RCDP are removed, preferably about 90% or more of the RCDP are removed.

18. A method according to any one of claims 1 to 17, wherein the modified plasma has 90% or less, 80% or less, 70% or less, 60% or less, 50% or less, 40% or less, 30% or less, 20% or less, 10% or less RCDP compared to the plasma, preferably 10% or less RCDP compared to the plasma.

19. A method according to any one of claims 1 to 18, wherein the modified plasma is substantially free of RCDP.

20. A method according to any one of claims 1 to 19, wherein the modified plasma has a clotting time at least two times longer than the plasma, preferably at least three times longer than the plasma, most preferably at least five times longer than the plasma.

21. A method according to any one of claims 1 to 20, wherein the modified plasma has a clotting time approximately the same as the clotting time for control plasma from a healthy subject.

22. A method according to any one of claims 1 to 21, wherein the plasma is isolated from a subject with a haemoglobinopathy, haemolytic anaemia and/or an erythrocyte membrane disorder.

23. A method according to any one of claims 2 to 22, wherein the haemoglobinopathy is sickle cell disease, preferably wherein the sickle cell disease is sickle cell anaemia, HbSβ-thalassemia, or HbSC disease, more preferably wherein the sickle cell disease is sickle cell anaemia.

24. A method according to any one of claims 1 to 23, wherein the plasma is platelet-free plasma and/or the plasma is fresh plasma or fresh frozen plasma.

25. Modified plasma obtained or obtainable by a method according to any one of claims 1 to 24.

26. Plasma which is substantially free of RCDP which express the extracellular domain of GPA on their surface and have a median diameter of 6-9 μm or about 7 μm.

27. Plasma according to claim 26, wherein the plasma is fresh plasma or fresh frozen plasma.

28. An affinity column comprising a stationary phase which specifically binds one or more markers expressed on the surface of RCDP.

29. An affinity column according to claim 28 wherein the one or more markers are from the extracellular and/or intracellular membrane surface of erythrocytes or reticulocytes, preferably the one or more markers are from the extracellular membrane surface of erythrocytes or reticulocytes.

30. An affinity column according to claim 28 or claim 29 wherein the one or more markers are selected from the list consisting of: GPA, AE1, GLUT1, GPC, and PS.

31. An affinity column according to any one of claims 28 to 30 wherein the marker is the extracellular and/or cytoplasmic domain of GPA.

32. An affinity column according to any one of claims 28 to 31 wherein the molecule is an antibody or an antibody fragment, preferably an antibody.

33. An apheresis device or an apheresis kit comprising a molecule or a bead which specifically binds one or more antigens markers expressed on the surface of RCDP, and/or an affinity column according to any one of claims 28 to 32.

34. Use of a molecule or a bead which specifically binds one or more antigens markers expressed on the surface of RCDP, an affinity column according to any one of claims 28 to 32, an apheresis device or an apheresis kit according to claim 33, for removing RCDP, for decreasing hypercoagulability, and/or for increasing plasma clotting time.

35. Use of a molecule or a bead which specifically binds one or more antigens markers expressed on the surface of RCDP, an affinity column according to any one of claims 28 to 32, or an apheresis device or an apheresis kit according to claim 33, in a method according to any one of claims 1 to 24.

36. A method according to any one of claims 1 to 24, or use according to claim 34 or claim 35, wherein the plasma clotting time is the Factor Xa-activated clotting time, the thrombin time, the activated partial thromboplastin time, the prothrombin time, or the activated clotting time, preferably the plasma clotting time is the Factor Xa-activated plasma clotting time.

37. A method according to any one of claims 1 to 24 or claim 36, or use according to any one of claims 34 to 36, wherein the plasma clotting time is increased by at least two-fold, at least three-fold, or at least five-fold.
